(19) United States
(12) Patent Application Publication
(10) Pub. No.: US 2024/0382324 A1
(43) Pub. Date: Nov. 21, 2024

(54) INTRAVASCULAR STENT

(71) Applicant: Abbott Cardiovascular Systems Inc., Santa Clara, CA (US)

(72) Inventors: Alan Hughes, Kilkenny (IE); Diarmuid Wall, Clonmel (IE); Diem Uyen Ta, San Jose, CA (US)

(73) Assignee: Abbott Cardiovascular Systems Inc., Santa Clara, CA (US)

(21) Appl. No.: 18/633,933

(22) Filed: Apr. 12, 2024

Related U.S. Application Data
(60) Provisional application No. 63/467,198, filed on May 17, 2023.

(51) Int. Cl. A61F 2/89

(52) U.S. Cl. CPC A61F 2/89

(57) ABSTRACT

An expandable stent for implantation in a body lumen, such as an artery, consists of a plurality of radially expandable cylindrical rings generally aligned on a common longitudinal stent axis and interconnected by one or more interconnecting links placed so that the stent is flexible in the longitudinal direction. The radial strength of the stent is enhanced by selectively increasing the width of the crests by offsetting the center points of inner arcs relative to outer arcs.

### CROSS-REFERENCES TO RELATED APPLICATION

This application is a non-provisional application of U.S. Provisional Application No. 63/467,198 filed May 17, 2023, incorporated by reference in its entirety.

### BACKGROUND

### Background

The present invention relates to expandable endoprosthesis devices, generally known as stents, which are designed for implantation in a patient's body lumen, such as a blood vessel to maintain the patency thereof. These devices are particularly useful in the treatment and repair of blood vessels after a stenosis has been compressed by percutaneous transluminal coronary angioplasty (PTCA) or percutaneous transluminal angioplasty (PTA) or removed by atherectomy or other means such as direct stenting.

Stents are generally cylindrically-shaped devices which function to hold open and sometimes expand a segment of a blood vessel or other lumen such as a coronary artery. They are particularly suitable for use to support the lumen or hold back a dissected arterial lining which can occlude the fluid passageway therethrough.

A variety of devices are known in the art for use as stents and has included a plastically deformable wire mesh in a variety of patterns that is expanded after being placed intraluminally on a balloon catheter; helically wound coiled springs manufactured from an expandable heat sensitive metal; and self-expanding stents inserted in a compressed state and shaped in a zigzag pattern. One of the difficulties encountered using prior art stents involved maintaining the radial rigidity needed to hold open a body lumen while at the same time maintaining the longitudinal flexibility of the stent to facilitate its delivery and accommodate the often tortuous path of the body lumen. Other problems encountered by using prior art stents involve maintaining stent longitudinal flexibility and longitudinal stent compression. Some prior art stents are highly longitudinally flexible, however, these stents tend to experience higher longitudinal stent compression when the stent is subject to an axial load.

Another problem area for prior art stents has been the flexibility in the stent distal end. Many prior art stents have uniform longitudinal flexibility along their lengths. It may be desirable to have a stent with a higher degree of flexibility in the distal end to better track through tortuous calcified anatomy.

The purpose of a cardiovascular stents is to maintain lumen area post deployment, to achieve this a stent needs to be able to reach a lesion, characterized by its deliverability and maintain the desired lumen diameter by resisting the compressive forces of a vessel.

Typical vessels that require revascularization range from 2 mm to 5 mm in diameter (Source) with the majority of vessels being 2.5 mm to 3.5 mm in diameter. Clinical evidence shows that stent cross sectional area which can be defined as minimum stent area (MSA) is an independent predictor of target vessel revascularization. Evidence also shows that both fibrotic lesions and calcified lesions contribute to poor stent expansion.

Commercial stents having nominal diameters of 3.0 mm typically have a radial strength of ˜1200 to 1700 mmHG. Indications from OCT analysis of vessels indicate that higher radial strength is needed to meet the needs of patients with resistant lesions.

Radial strength is typically achieved by adding more support to the stent. However, this is a complex balancing act between maintaining strut thickness, an appropriate surface area for drug carrying and deliverability such that a higher radial strength stent can reach the resistant lesions.

The devices disclosed herein overcome the deficiencies of the prior art devices and provide stents having a high degree of longitudinal flexibility, increased radial strength, and improved longitudinal strength compression.

### SUMMARY OF THE INVENTION

In the embodiments disclosed herein, the focus is to increase radial strength without sacrificing desirable features such as longitudinal flexibility or impacting longitudinal stent compression. A two-link design connecting multiple rings together allows for a larger crest strut width (SW) without increasing theoretical minimum crimp (TMC), thereby increasing radial strength (RS). Importantly, the design of offset inner arcs allows the crest strut width to increase at the crest apex without increasing TMC, consequently increasing radial strength. Further, the design of the offset inner arcs allows crest modification to reduce stress and strain at the crest, with a minimal impact on radial strength. By adjusting strut dimension tolerancing in manufacturing, the stress and stain at the crests is reduced. Finally, by providing a three-link pattern in the stent proximal end, the longitudinal stent compression is improved while still maintaining the desired longitudinal flexibility provided by the two-link pattern.

The present devices are directed to stents having increased radial strength, enhanced longitudinal flexibility, and high longitudinal strength compression while maintaining the same number of rings per unit length. The stents have greater flexibility along their longitudinal axis to facilitate delivery through tortuous body lumens but remain resistant to longitudinal compression incurred when another device tries to cross the deployed stent. The unique link patterns and optimal strut widths of the stents permit both greater longitudinal flexibility and higher longitudinal strength compression compared to prior art stents.

Each of the different embodiments of stents of the present invention includes a plurality of adjacent cylindrical rings which are generally expandable in the radial direction and arranged in alignment along a longitudinal stent axis. The cylindrical rings are formed in a serpentine wave pattern transverse to the longitudinal axis and contain a plurality of alternating peaks and valleys. At least one link extends between adjacent cylindrical rings and connects them to one another. These links insure minimal longitudinal contraction during radial expansion of the stent in the body vessel. The links can be positioned in differing configurations or patterns along the stent length to enhance longitudinal stent flexibility and enhance longitudinal strength compression.

In one embodiment disclosed herein, a stent has a tubular body having a distal end ring, a proximal end ring, and a plurality of body rings therebetween. The distal end ring, the proximal end ring, and the body rings are aligned in an in-phase configuration. The proximal end ring is connected to a first proximal body ring by three links, and the first proximal body ring is connected to a second proximal body ring by three links. The distal end ring and the plurality of body rings up to the second proximal body ring are all connected by two links. Each of the distal end ring, proximal end ring, and the plurality of body rings has six crests. Each of the distal end ring and the plurality of body rings has four long crests and two short crests. The four long crests have an outer arc and an inner arc, wherein a center point of the inner arc is offset from a center point of the outer arc in a direction away from the inner arc, thereby forming a non-uniform width on the four long crests. The two short crests also have an outer arc and an inner arc, wherein a center point of the inner arc is offset from a center point of the outer arc in a direction away from the inner arc, thereby forming a non-uniform width on the short crests. For a stent that is 18 mm long, the number of links connecting the rings has a pattern of 2-2-2-2-2-2-2-2-2-2-3-3 moving from the distal end ring toward the proximal end ring. In one embodiment, the variation in offset between the center point of the inner arc and the center point of the outer arc is in a range from 2 μm to 100 μm, wherein the inner arc and the outer arc have an arc configuration of a semi-circle arc, or a polyline arc, or a 3-point arc.

Each of the stents of the present invention can be readily delivered to the desired luminal location by mounting it on an inflatable member, such as a balloon of a delivery catheter, and passing the catheter-stent assembly through the body lumen to the implantation site. A variety of means for securing the stent to the inflatable member of the catheter for delivery to the desired location is available. It is presently preferred to compress or crimp the stent onto the uninflated balloon in a known manner.

Other features and advantages of the present invention will become more apparent from the following detailed description of the invention, when taken in conjunction with the accompanying exemplary drawings.

### BRIEF DESCRIPTION OF THE DRAWINGS

FIG. 1 is an elevational view of a prior art stent, partially in sections, depicting a stent mounted on a delivery catheter disposed within a vessel.

FIG. 2 is an elevational view of a prior art stent, partially in section, similar to the stent of FIG. 1, wherein the stent is expanded within a vessel.

FIG. 3 is an elevational view of a prior art stent, partially in section, showing the stent of FIG. 1 expanded within a vessel after withdrawal of the delivery catheter.

FIG. 4A is a plan view of a prior art stent depicting one embodiment of the stent in a flattened configuration and illustrating the connecting link pattern between the end rings and the body rings.

FIG. 4B is a perspective view of the stent of FIG. 4A in a tubular configuration.

FIG. 5A is a plan view of the present invention stent depicting a specific link pattern between adjacent rings.

FIG. 5B is an elevational view in perspective depicting the stent of FIG. 5A in a cylindrical form.

FIG. 6 is an enlarged view of a crest section of a prior art stent depicting a common center point for the outer arc and the inner arc.

FIG. 7 is an enlarged view of a crest section of the stent of the present invention depicting an inner arc center point offset from an outer arc center point.

FIG. 8 is a plan view of a portion of the stent of FIG. 5 depicting a short crest, a long crest, a Y-crest, and a W-crest.

FIG. 9 is a plan view of a portion of the proximal end ring of the stent of FIG. 5 depicting a proximal U-crest and a proximal Y-crest.

FIG. 10 is a plan view of a portion of a short crest depicting the center point of the inner arc being offset from the center point of the outer arc.

FIG. 11 is a plan view of the W-crest, the Y-crest and the short crest depicting the center points of the inner arcs being offset from the center points of the outer arcs.

FIG. 12 is a plan view of a portion of the W-crest and the short crest depicting the offset inner arcs.

FIG. 13 is a graph comparing the radial strength of a stent having a uniform crest width along the inner arc and the outer arc and a stent having the center point of an inner arc offset from the center point of an outer arc thereby forming a non-uniform crest width.

FIG. 14 is a graph comparing the radial strength of commercially available stents with the present invention stent having the center point of the inner arc offset from the center point of the outer arc.

FIG. 15 is a graph comparing the radial strength of commercially available stents with the present invention stents.

FIG. 16 is an elevational view of two stents of the invention depicting varying degrees of overlap created during finite element analysis testing.

FIG. 17 is a graph comparing the longitudinal compression of commercially available stents with the present invention stents.

FIG. 18 is an elevational view comparing the longitudinal compression of commercially available stents with the present invention stents having the center point of the inner arc offset from the center point of the outer arc.

FIGS. 19A and 19B are an enlarged view of a crest section of the stent depicting an inner arc center point offset away from and up or down from the center point of the outer arc center point.

FIG. 20A is a plan view of the present invention stent depicting a specific link pattern between adjacent rings.

FIG. 20B is a plan view of a portion of the stent of FIG. 20A depicting a plurality crests.

FIG. 21 is an enlarged view of a crest section depicting an outer arc being increased to form an outer conehead shape resulting in an increase in the crest profile.

FIG. 22 is an enlarged view of a crest section depicting an outer arc being increased at only the crest apex.

FIG. 23 is an enlarged view of a crest section depicting an inner arc being offset inwardly thereby forming an inner conehead.

FIG. 24 is a graph depicting the radial strength of two prototype stents.

FIG. 25 is an enlarged view of a crest section depicting a reduced inner radii along the sides of the inner arc.

### DETAILED DESCRIPTION OF THE PREFERRED EMBODIMENTS

### Prior Art Stents

Prior art stent designs, such as the MULTILINK STENT® manufactured by Abbott Cardiovascular Systems, Inc., Santa Clara, Calif., include plurality of cylindrical rings that are connected by three connecting members between adjacent cylindrical rings. Each of the cylindrical rings is formed of a repeating pattern of U-, Y-, and W-shaped members, typically having three repeating patterns forming each cylindrical ring. A more detailed discussion of the configuration of the MULTILINK STENT® can be found in U.S. Pat. No. 5,569,295 (Lam) and U.S. Pat. No. 5,514,154 (Lau et al.), whose contents are hereby incorporated by reference.

Prior art stent structure is shown in FIGS. 1-4B, where a stent is mounted onto a delivery catheter. The stent generally includes a plurality of radially expandable cylindrical rings disposed generally coaxially and interconnected by links disposed between adjacent cylindrical rings. The delivery catheter has an inflatable portion or balloon for expanding the stent within an artery or other vessel.

The delivery catheter onto which the stent is mounted is similar to a conventional balloon dilatation catheter for angioplasty procedures. The balloon may be formed of suitable materials such as polyethylene, polyethylene terephthalate, polyvinyl chloride, nylon, and ionomers such as SURLYN® manufactured by the Polymer Products Division of the DuPont Company. Other polymers also may be used.

In order for the stent to remain in place on the balloon during delivery to the artery, the stent is compressed or crimped onto the balloon.

The delivery of the stent to a coronary artery, for example, is accomplished in the following manner. The stent is first mounted onto an inflatable balloon on the distal extremity of the delivery catheter. The stent may be crimped down onto the balloon to obtain a low profile. The catheter-stent assembly can be introduced within the patient's vasculature in a conventional technique through a guiding catheter (not shown). A guidewire is advanced through the arterial vasculature. The catheter-stent assembly is then advanced over the guidewire within artery. The balloon of the catheter is inflated to expand the stent against the inside of the artery, which is illustrated in FIG. 2. While not shown in the drawing, the artery is preferably over expanded slightly by the expansion of the stent to seat or otherwise embed the stent to prevent movement. Indeed, in some circumstances during the treatment of stenotic portions of an artery, the artery may have to be expanded considerably in order to facilitate passage of blood or other fluid therethrough.

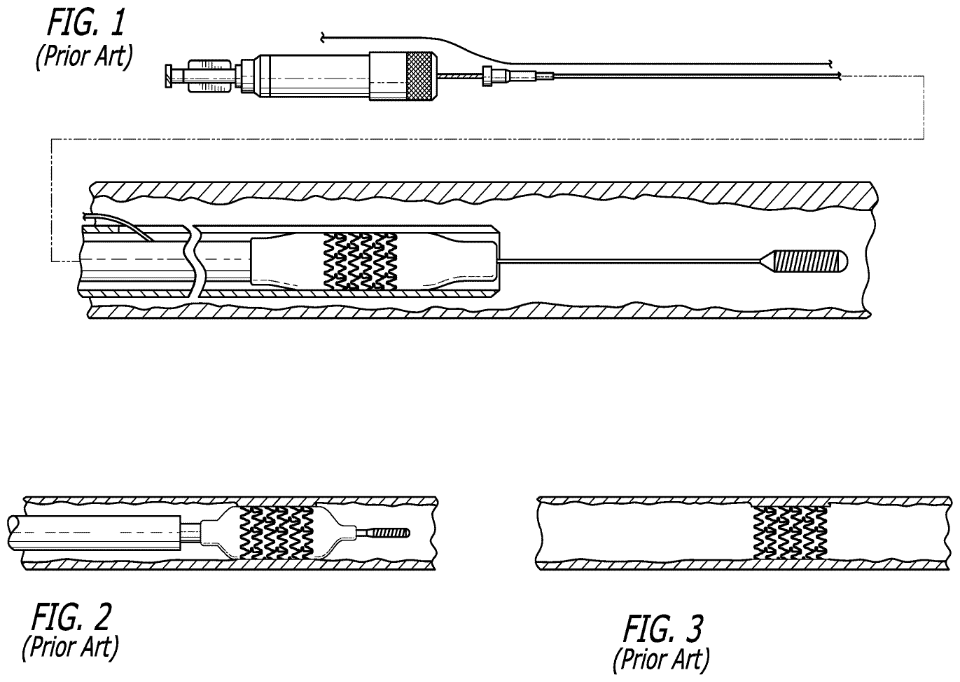

In general, the stent serves to hold open the artery after catheter is withdrawn, as illustrated in FIG. 3. The undulating component of the cylindrical elements of the stent is relatively flat in a transverse cross-section so that when the stent is expanded, the cylindrical rings are pressed into the wall of the artery. The cylindrical rings of the stent that are pressed into the wall of the artery will eventually be covered with endothelial cell growth that further minimizes blood flow turbulence. The serpentine pattern of the cylindrical rings provides good tacking characteristics to prevent stent movement within the artery.

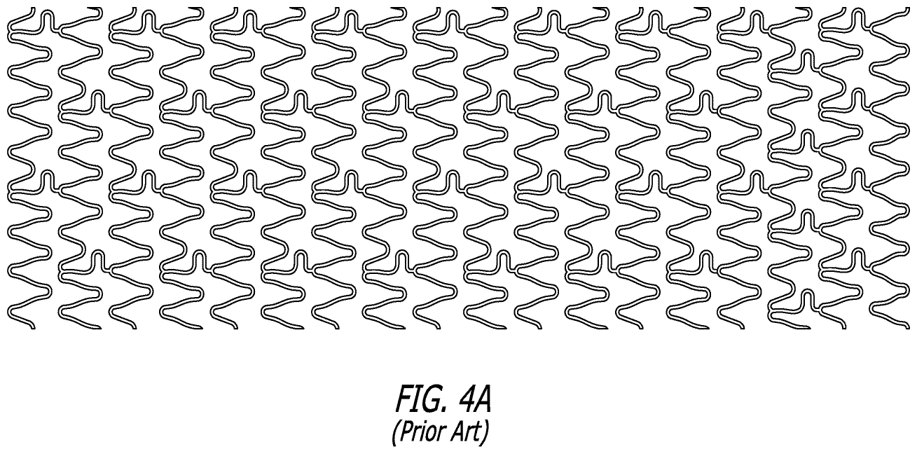

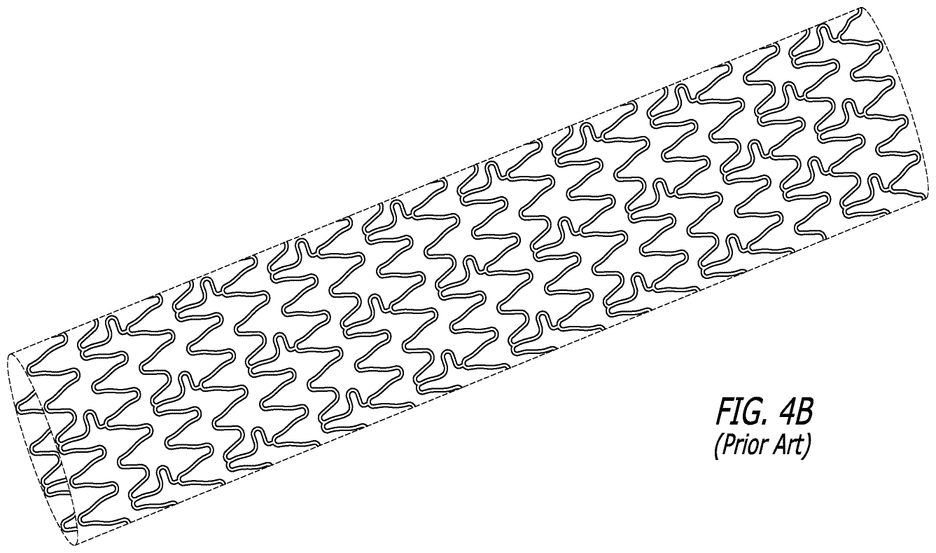

In FIGS. 4A and 4B, the prior art stent has eighteen eight-crest (or peaks) rings that are positioned in an in-phase relationship and are connected by links. The tubular body has a compressed diameter and an expanded implanted diameter. The link configuration provides several portals for enhanced side branch vessel access and maximum flexibility on the distal end and mid-portion and maximum stability on the proximal end. The tubular body has a distal end, mid-portion, and a proximal end. Basically, in this embodiment, the distal end ring and the body rings are all connected by two links. The two most proximal body rings and the proximal end ring are all connected by four links. The links extend from a valley of one ring to a peak of the adjacent ring. All of the rings have six peaks or crests. The rings with four connecting links provide structure to the design to minimize any compression under longitudinal forces, while the rings with two connecting links reduce the chance of the side branch vessel being blocked by a link by one-third.

### High Radial Strength Stent

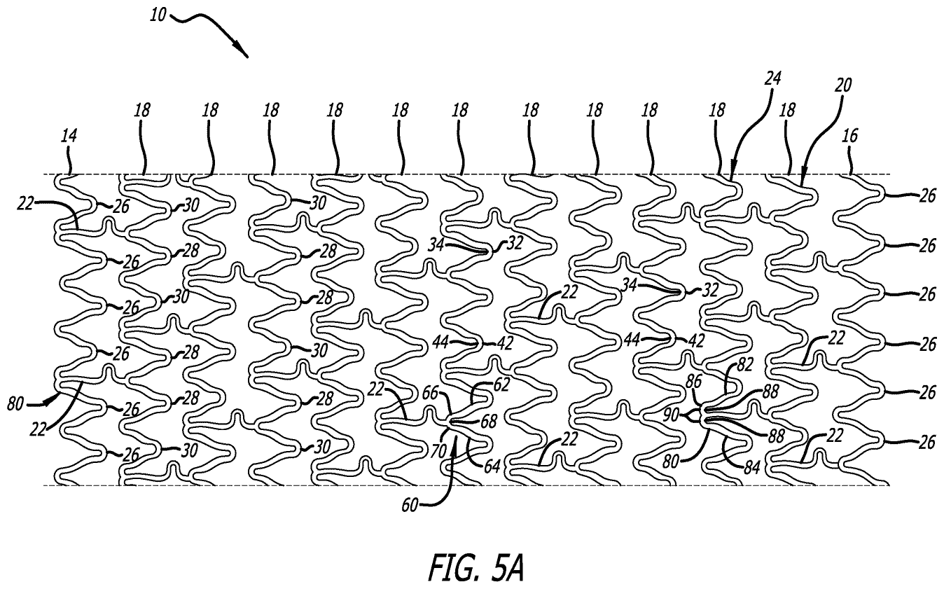

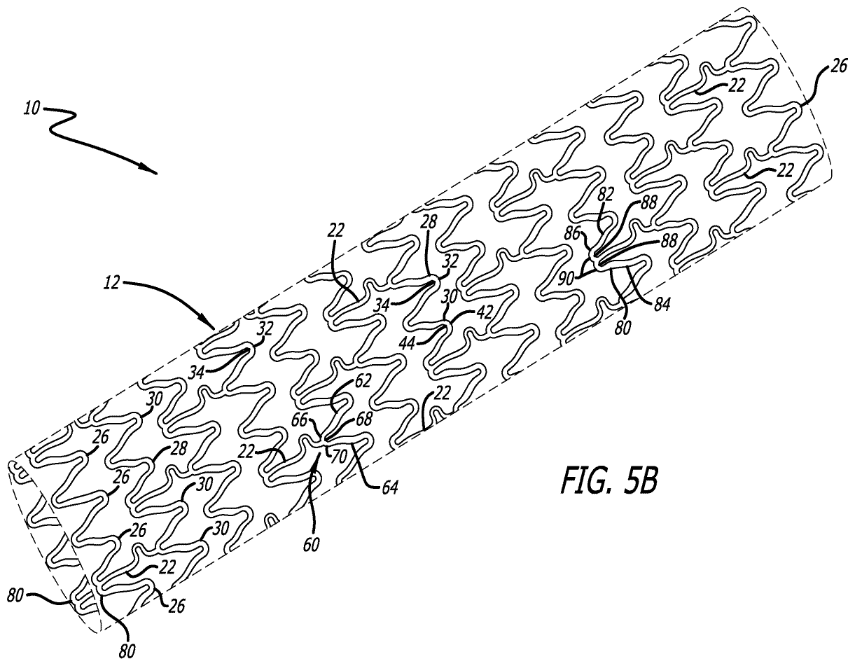

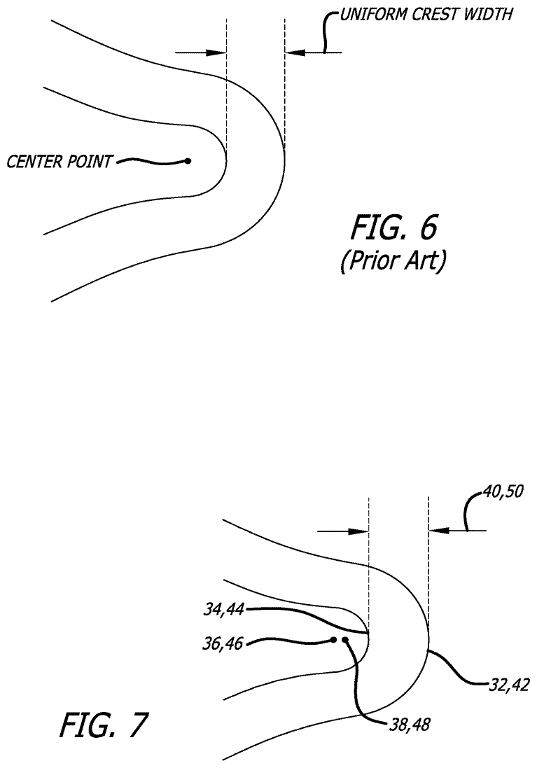

As shown in FIG. 5, the stent 10 of the preset invention has been designed to increase radial strength over prior art stents and provide best-in-class deliverability. Critical design factors (discussed further wherein) were considered and include:
High radial strength without exceeding material limits (for example CoCr) and compromising ring spacing/overall stent length, strut thickness, and profile.
Improved flexibility and conformality.

All of the foregoing factors and design parameters have been incorporated into the stent embodiments disclosed herein.

Design features such as crest width, bar arm width, bar arm length, inner crest radii and ring spacing are the main critical factors that contribute to stent designs. The present invention development analyzed and tested unique features that contribute to an improvement in radial strength over any other stent design on the market today.

One critical design feature in the design of the present invention is an offset inner crest design. This feature creates a larger strut width at the apex of the crest of the stent by adding an offset arc to the inner crest as opposed to a concentric arc feature typically associated with commercial stent designs. This feature allows maintenance of the number of rings per unit length and maintains the ring spacing which allows balancing the radial strength, scaffolding and deliverability.

In one embodiment disclosed herein, as shown in FIGS. 5A, 5B and 7, a stent 10 has a tubular body 12 having a distal end ring 14, a proximal end ring 16, and a plurality of body rings 18 therebetween. The distal end ring 14, the proximal end ring 16, and the body rings 18 are aligned in an in-phase configuration. The proximal end ring 16 is connected to a first proximal body ring 20 by three links 22, and the first proximal body ring 20 is connected to a second proximal body ring 24 by three links 22. The distal end ring 14 and the plurality of body rings 18, up to the second proximal body ring 24, are all connected by two links 22. Each of the distal end ring 14, proximal end ring 16, and the plurality of body rings 18, has six crests 26. The six crests 26 may sometimes be referred to herein as U-shaped members 26 because the crest structure resembles a U-shape. Each of the distal end ring 14 and the plurality of body rings 18 has long crests 28 (U-shaped members) and short crests 30 (U-shaped members). The long crests 28 have an outer arc 32 and an inner arc 34, wherein a center point 36 of the inner arc 34 is offset from a center point 38 of the outer arc 32 in a direction away from the inner arc 34, thereby forming an increased non-uniform crest width 40 on the long crests 28. The short crests 30 also have an outer arc 42 and an inner arc 44, wherein a center point 46 of the inner arc 44 is offset from a center point 48 of the outer arc 42 in a direction away from the inner arc 44, thereby forming an increased non-uniform width 50 on the short crests 30. The number of links 22 connecting adjacent rings has a pattern of 2-2-2-2-2-2-2-2-2-2-3-3 moving from the distal end ring 14 toward the proximal end ring 16. In one embodiment, the variation in offset between the center point 36, 46 of the inner arc 34, 44 and the center point 38, 48 of the outer arc 32, 42 is in a range from 2 μm to 100 μm, wherein the inner arc 34, 44 and the outer arc 32, 42 have an arc configuration of a semi-circle arc, or a polyline arc (multiple arcs with multiple center points), or a 3-point arc. In general, it is well known in the art that the two-link 22 connections enable greater flexibility of the stent in the expanded and crimped configurations as compared to three-link connections and higher. The two-link 22 connections also enable the ability of the individual crest dimensions (e.g., an increased non-uniform crest width 40, 50) within a ring to increase within the same circumferentially crimped profile. In other words, the two-link 22 connections allow the crest strut width to increase without increasing the theoretical minimum crimp (TMC) outer diameter. This is done to increase radial strength. Moving the inner arc center point 34, 44 are added to further increase radial strength. Moving the inner arc center point 34, 44 can be added to any link connection configuration to increase radial strength without increasing TMC. The three links 22 on the proximal section of the stent 10 creates greater stability for the stent 10 in the event of a linear force compressing on the proximal end of the stent 10.

In another embodiment disclosed herein, as shown in FIGS. 5A, 5B and 8-11, a stent 10 has a tubular body 12 having a distal end ring 14, a proximal end ring 16, and a plurality of body rings 18 therebetween. The distal end ring 14, the proximal end ring 16, and the body rings 18 are aligned in an in-phase configuration. The proximal end ring 16 is connected to a first proximal body ring 20 by three links 22, and the first proximal body ring 20 is connected to a second proximal body ring 24 by three links 22. The distal end ring 14 and the plurality of body rings 18, up to the second proximal body ring 24, are all connected by two links 22. Each of the distal end ring 14, proximal end ring 16, and the plurality of body rings 18, has six crests 26. The six crests 26 may sometimes be referred to herein as U-shaped members 26 because the crest structure resembles a U-shape. Each of the distal end ring 14 and the plurality of body rings 18 has long crests 28 (U-shaped members) and short crests 30 (U-shaped members). The long crests 28 have an outer arc 32 and an inner arc 34, wherein a center point 36 of the inner arc 34 is offset from a center point 38 of the outer arc 32 in a direction away from the inner arc 34, thereby forming an increased non-uniform crest width 40 on the long crests 28. The short crests 30 also have an outer arc 42 and an inner arc 44, wherein a center point 46 of the inner arc 44 is offset from a center point 48 of the outer arc 42 in a direction away from the inner arc 44, thereby forming an increased non-uniform width 50 on the short crests 30. The number of links 22 connecting adjacent rings has a pattern of 2-2-2-2-2-2-2-2-2-2-3-3 moving from the distal end ring 14 toward the proximal end ring 16. In one embodiment, the variation in offset between the center point 36, 46 of the inner arc 34, 44 and the center point 38, 48 of the outer arc 32, 42 is in a range from 2 μm to 100 μm, wherein the inner arc 34, 44 and the outer arc 32, 42 have an arc configuration of a semi-circle arc, or a polyline arc, or a 3-point arc. In this embodiment, each of the plurality of body rings 18, the first proximal body ring 20, the second proximal body ring 24, and the proximal end ring 16 has a Y-shaped member 60. The Y-shaped members 60 are so described because they resemble a Y-shape having a first bar arm 62, a second bar arm 64, a Y-crest 66, an inner arc 68, an outer arc 70, and link 22 extending from the outer arc 70. The Y-crest is formed by the inner arc 68 and the outer arc 70. In order to increase the radial strength of the Y-shaped members 60, a center point 72 of the inner arc 68 is offset from a center point 74 of the outer arc 70 in a direction away form the inner arc 68, thereby forming an increased non-uniform crest width 76 on the Y-shaped members 60. In general, it is well known in the art that the two-link 22 connections enable greater flexibility of the stent in the expanded and crimped configurations as compared to three-link connections and higher. The two-link 22 connections also enable the ability of the individual crest dimensions (e.g., an increased non-uniform crest width 76) within a ring to increase within the same circumferentially crimped profile. In other words, the two-link 22 connections allow the crest strut width to increase without increasing the theoretical minimum crimp (TMC) outer diameter. This is done to increase radial strength. Moving the inner arc center point 34, 44 are added to further increase radial strength. Moving the inner arc center point 34, 44 can be added to any link connection configuration to increase radial strength without increasing TMC. The three links 22 on the proximal section of the stent 10 creates greater stability for the stent 10 in the event of a linear force compressing on the proximal end of the stent 10.

In one embodiment disclosed herein, as shown in FIGS. 5A, 5B and 8-11, a stent 10 has a tubular body 12 having a distal end ring 14, a proximal end ring 16, and a plurality of body rings 18 therebetween. The distal end ring 14, the proximal end ring 16, and the body rings 18 are aligned in an in-phase configuration. The proximal end ring 16 is connected to a first proximal body ring 20 by three links 22, and the first proximal body ring 20 is connected to a second proximal body ring 24 by three links 22. The distal end ring 14 and the plurality of body rings 18, up to the second proximal body ring 24, are all connected by two links 22. Each of the distal end ring 14, proximal end ring 16, and the plurality of body rings 18, has six crests 26. Each of the distal end ring 14 and the plurality of body rings 18 has long crests 28 and short crests 30. The long crests 28 have an outer arc 32 and an inner arc 34, wherein a center point 36 of the inner arc 34 is offset from a center point 38 of the outer arc 32 in a direction away from the inner arc 34, thereby forming an increased non-uniform crest width 40 on the long crests 28. The short crests 30 also have an outer arc 42 and an inner arc 44, wherein a center point 46 of the inner arc 44 is offset from a center point 48 of the outer arc 42 in a direction away from the inner arc 44, thereby forming an increased non-uniform width 50 on the short crests 30. The number of links 22 connecting adjacent rings has a pattern of 2-2-2-2-2-2-2-2-2-2-3-3 moving from the distal end ring 14 toward the proximal end ring 16. In one embodiment, the variation in offset between the center point 36, 46 of the inner arc 34, 44 and the center point 38, 48 of the outer arc 32, 42 is in a range from 2 μm to 100 μm, wherein the inner arc 34, 44 and the outer arc 32, 42 have an arc configuration of a semi-circle arc, or a polyline arc, or a 3-point arc. In this embodiment, each of the distal end rings 14, the plurality of body rings 18, the first proximal body ring 20, and the second proximal body ring 24 has a W-shaped member 80. The W-shaped members 80 are so described because they resemble a W-shape, having a first bar arm 82, a second bar arm 84, and link 22 extending therebetween. The W-shaped members 80 have a W-crest 86 having an inner arc 88 and an outer arc 90, the link 22 extending from the inner arc 88. In order to increase the radial strength of the W-shaped members 80, a center point 102 of the inner arc 88 is offset from a center point 100 of the outer arc 90 in a direction away from the inner arc 88, thereby forming an increased non-uniform crest width 104 on the W-shaped members 80. In general, it is well known in the art that the two-link 22 connections enable greater flexibility of the stent in the expanded and crimped configurations as compared to three-link connections and higher. The two-link 22 connections also enable the ability of the individual crest dimensions (e.g., an increased non-uniform crest width 104) within a ring to increase within the same circumferentially crimped profile. The three-link 22 on the proximal section of the stent 10 creates greater stability for the stent 10 in the event of a linear force compressing on the proximal end of the stent 10.

For all of the embodiments disclosed herein, the stent length can range from 8 mm to 58 mm. For the stent length in this range, the link pattern will always consist of two links 22 between the distal end ring 14 and all of the plurality of body rings 18 up to the second proximal body ring 24, wherein there are three links 22 between the second proximal body ring 24 and the first proximal body ring 20, and three links 22 between the fist proximal body ring 20 and the proximal end ring 16. Thus, some embodiments will have link patterns of 2-2-2-3-3; 2-2-2-2-3-3; 2-2-2-2-2-3-3; 2-2-2-2-2-2-3-3; 2-2-2-2-2-2-2-3-3; 2-2-2-2-2-2-2-2-3-3; 2-2-2-2-2-2-2-2-2-3-3; 2-2-2-2-2-2-2-2-2-2-3-3; and 2-2-2-2-2-2-2-2-2-2-N-3-3. In the last embodiment, the “Nth” link represents any number of two-link connections necessary for the specific length of the stent from 8 mm long up to 58 mm long.

As disclosed herein, when reference is made to the center point 36, 46, 72 and 102 of the inner arc 34, 44, 68 and 88 (respectively) being offset in a direction away from the inner arc, the direction is away from the concave section of the inner arc as shown in FIGS. 7, 10 and 11.

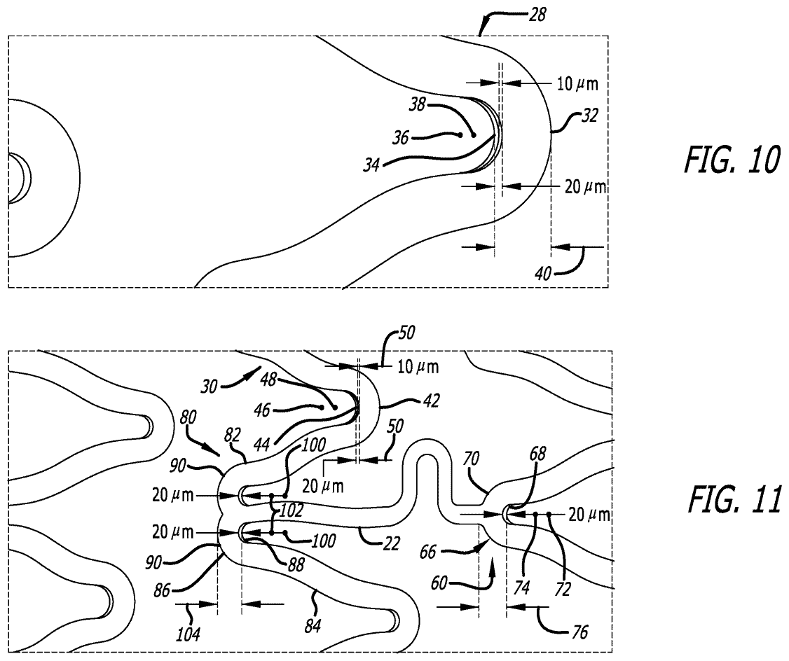

As shown in Table 1, the widths of the various crests described herein can vary in the range from 40 μm to 250 μm and preferably in the range from 80 μm to 145 μm. Importantly, as shown in Table 1 and FIGS. 10-12, the amount the center point (36, 72, 102) of the inner arc (34, 68, 88 respectively) is offset from the center point (38, 74, 100) of the outer arc (32, 70, 90 respectively) can vary in the range from 2 μm to 100 μm, and preferably in the range from 5 μm to 25 μm. For example, as shown in FIG. 10, a long crest 28 has an outer arc 32 and an inner arc 34, wherein a center point 36 of the inner arc 34 is offset from a center point 38 of the outer arc 32 by either 10 μm or 20 μm in a direction away from the concave portion of the inner arc 34, thereby forming a non-uniform increased crest width 40.

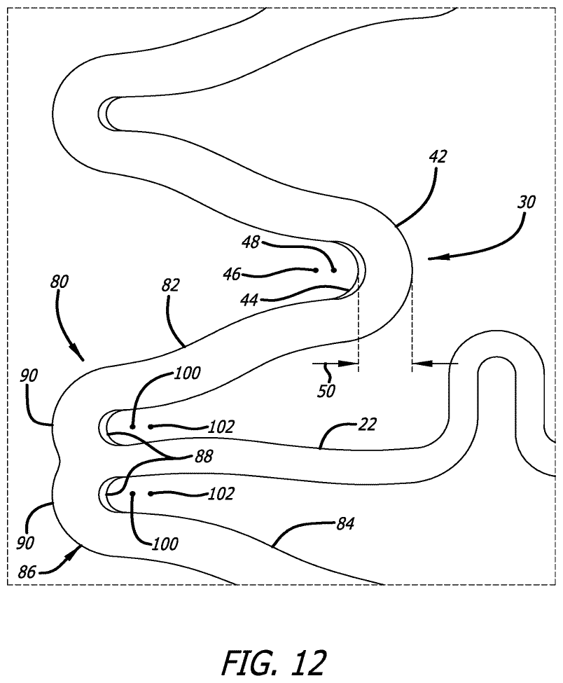

Similar to FIG. 10, the embodiments shown in FIGS. 11 and 12 disclose offset center points on the inner arcs of the short crest 30, the Y-shaped member 60, and the W-shaped member 80. The short crest 30 has an outer arc 42 and an inner arc 44. A center point 46 of the inner arc 44 is offset from a center point 48 of the outer arc 42 by either 10 μm or 20 μm in a direction away from the concave portion of the inner arc 44, thereby forming a non-uniform increased crest width 50. The Y-shaped member 60 has a first bar arm 62, a second bar arm 64, a Y-crest 66, an inner arc 68, an outer arc 70, and a link 22 extending from the outer arc 70. A center point 72 of the inner arc 68 is offset from a center point 74 of the outer arc 70 by 20 μm in a direction away from the concave portion of the inner arc 68, thereby forming a non-uniform increased crest width 76. The W-shaped member 80 has a first bar arm 82, a second bar arm 84, a W-crest 86, an inner arc 88, and an outer arc 90. A center point 102 of the inner arc 88 is offset from a center point 100 of the outer arc 90 by 20 μm in a direction away from the concave portion of the inner arc 88, thereby forming a non-uniform increased crest width 104.

In all of the embodiments of FIGS. 10-12, and as described in Table 1, the offset center point of the inner arc will increase the radial strength proportionally to the amount of the offset, thereby significantly increasing the radial strength of stent 10 without sacrificing crimp profile, flexibility and deliverability and maintaining the desired longitudinal compression.

TABLE 1

| Crest Location/Type | | | |
|---|---|---|---|
| Options For Offset | Crest Width | Inner Crest Offset | |
| Crest Application | Variations | Arc Variations | Arc Type |
| Crest Type | 80 μm, 90 μm, 100 μm, | 5 μm, 10 μm, 15 μm, | Single arc (semi- |
| Short Crest | 105 μm, 110 μm, | 20 μm, 25 μm | circle), |
| Long Crest | 115 μm, 125 μm, 130 | Any offset crest from | polyline arc, |
| U- Crest | μm, 135 μm, 140 μm, | 2 μm to 100 μm. | 3-point arc |
| W-crest (top) | 145 μm. | | |
| W-crest (bottom) | Any crest dimension | | |
| Y-Crest | between 40 μm- | | |
| Non-Linear Link | 250 μm | | |
| Crest | | | |
| Proximal Crest | | | |
| Proximal U-Crest | | | |
| Proximal Y-Crest | | | |

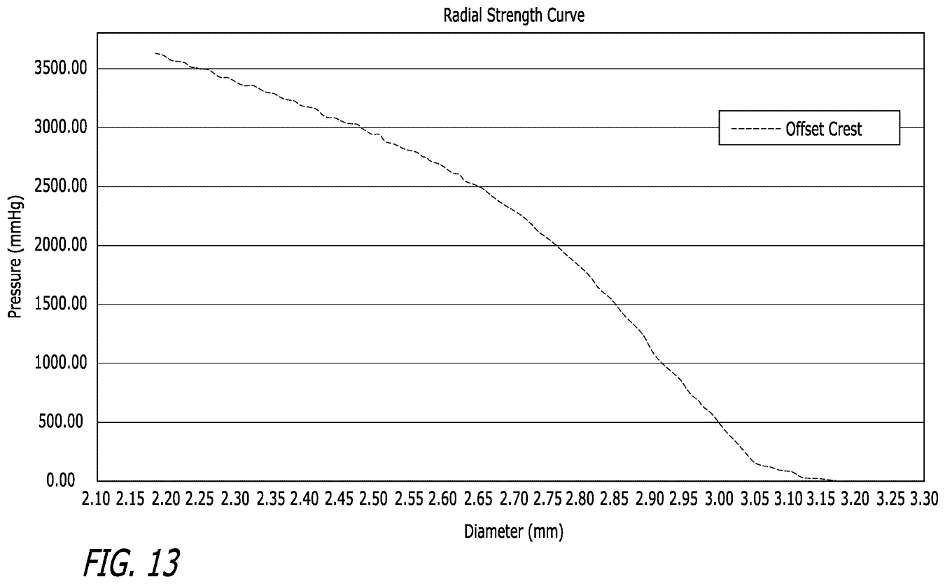

Referring to FIG. 13, radial strength curves are depicted of prototype stents of the embodiments disclosed herein having crests with an offset inner arc design. The offset inner arc of the present invention stent results in an increase in radial strength of the stent design, thus ensuring the structure has more resistance to radial compression from an artery. The radial strength curve of the offset inner arc design also shows an increased level of stiffness, indicated by the slope of the increasing portion of the curve in FIG. 13, while maintaining strength on a continued compression from 3.18 mm diameter to 2.18 mm. The radial strength curve shown in FIG. 13 was generated from data measured by the Abbott Test Method (STM 2056526 MSI RX550 Radial Test).

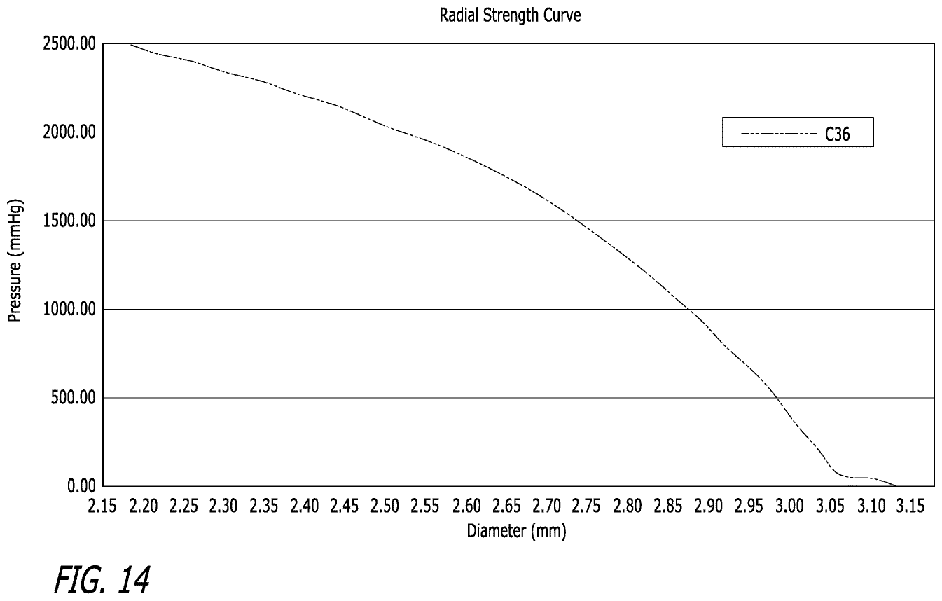

In FIG. 14, the radial strength curve of prototype stent C36 of the embodiments disclosed herein has crests with an offset inner arc design. The offset inner arc design coupled with other design feature modifications resulted in the C36 stent of the present invention having a significantly higher radial strength.

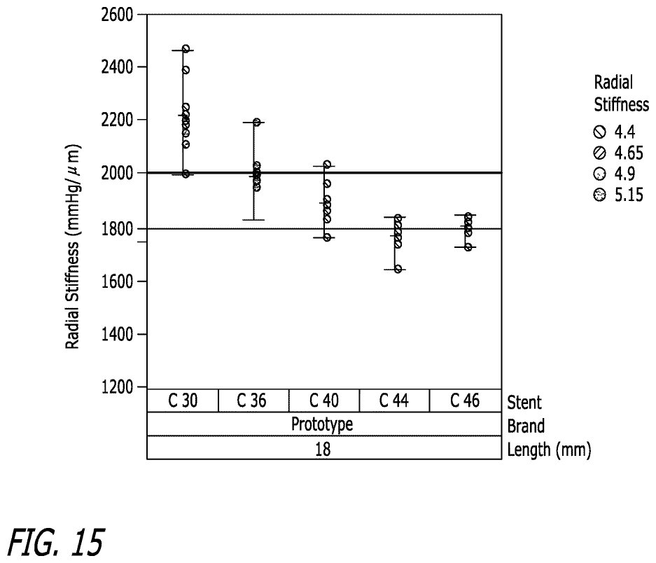

FIG. 15 and Table 2 show the radial stiffness of several prototypes (e.g., C36, C40, C44 and C46) of the present invention. The radial stiffness of all of the prototypes of the invention has increased. Table 2 includes data measuring stent radial strengths, FEA (PEEQ) or plastic strain and Von Mises Stress (VMS)) and radial stiffness. All of the data in Table 2 and FIG. 15 demonstrates the superior performance of the prototypes of the invention.

In one embodiment, disclosed in FIG. 15 and Table 2, prototype C36 shows superior radial strength (1994 mmHg) and radial stiffness (5.2 mmHG/μm) and is designed with offset inner arcs in only the U-shaped crests, long crests, and short crests. The C36 prototype stent 10 has U-shaped crests, U-shaped long crests 28 and U-shaped short crests 30 that have the center points 36, 46 of the inner arcs 34, 44 offset from the center points 38, 48 of the outer arcs 32, 42 in a direction away from the concave portion of the inner arcs. After testing prototype C36, the data shows the superior structural characteristics including radial strength 1994 (mmHg) and radial stiffness 5.2 (mmHg/μm). As tested, prototype C36 was made of L-605 cobalt-chromium, however, other materials known in the art to manufacture stents are contemplated.

TABLE 2

| | | | | Design | | | | |
|---|---|---|---|---|---|---|---|---|
| | | Number | Design | Features For | | | | |
| | | of Rings | Features | 2-Links | | VMS | | |
| Offset | | In 18 mm | 2-Link | Section: | PEEQ | (ksi) | Radial | Radial |
| Inner | Stent | Labeled | section: Strut | Inner Crest | (Max | (Max | Strength | Stiffness |
| Arc | Design | Length | Width (μm) | Radii (μm) | 90%) | 350 ksi) | (mmHg) | mm-Hg/μm |
| No | C30 | 13 | 125 crest width, | 50 U-crest | 97.8% | 350 | 2222 | 4.86 |
| | | | 145 bar arm | radii, | | | | |
| | | | width | 86 short | | | | |
| | | | | crest radii, | | | | |
| | | | | 57 long | | | | |
| | | | | crest radii | | | | |
| Yes | C33 | 12 | 135 crest width, | 50 U-crest | 99.0% | 350 | 2075 | 4.92 |
| | | | 145 bar arm | crest radii, | | | | |
| | | | width | 86 short | | | | |
| | | | | crest radii, | | | | |
| | | | | 57 long | | | | |
| | | | | crest radii | | | | |
| Yes | C36/C36* | 13 | 125 crest width, | 60 U-crest | 92.7% | 350/348* | 1994 | 5.22 |
| | | | 145 bar arm | radii, | 88.3%* | | | |
| | | | width | 96 short | | | | |
| | | | | crest radii, | | | | |
| | | | | 67 long | | | | |
| | | | | crest radii | | | | |
| Yes | C40 | 13 | 125 crest width, | 60 U-crest | 86.9% | 344 | 1894 | 4.78 |
| | | | 145 bar arm | radii, | | | | |
| | | | width | 96 short | | | | |
| | | | | crest radii, | | | | |
| | | | | 67 long | | | | |
| | | | | crest radii | | | | |
| Yes | C44 | 13 | 125 crest width, | 60 U-crest | 84.7% | 338 | 1771 | 4.76 |
| | | | (with exception | radii, | | | | |
| | | | of short crests) | 96 short | | | | |
| | | | (135 short crest | crest radii, | | | | |
| | | | width), 145 bar | 67 long | | | | |
| | | | arm width | crest radii | | | | |

In Table 2, the asterisk data represents the maximum dimensional conditions of +7.6 μm strut width and +6.6 μm strut thickness. Data with no asterisk represent the FEA dimensional conditions of +5.1 μm strut width and +4.4 μm strut thickness.

### Finite Element Analysis (FEA)

Numerous tests were conducted during the development of the stent embodiments herein, including FEA. Through various design iterations, FEA was conducted to determine the plastic strain (PEEQ) and Von Mises Stress (VMS) of the stent prototypes when they are exposed to manufacturing processes and deployment during use.
1. Crimp on to a balloon.
2. Recoil from a balloon after crimping.
3. Expansion of stent 1 (after balloon inflation).
4. Recoil of stent 1 (after balloon deflation).
5. Expansion of stent 2 (inner stent overlapped).
6. Recoil of stent 2 (after deflation of balloon).

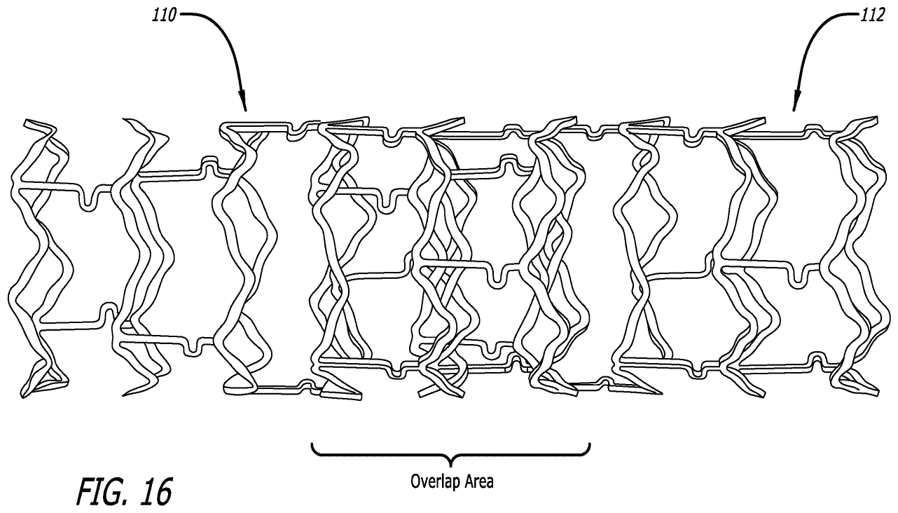

As shown in Table 3 and FIG. 16, FEA tests were conducted on the prototype stents of the present invention. In FIG. 16, outer stent 110 and inner stent 112 are overlapping (about three rings) and expanded to an inner diameter of 3.75 mm. Table 3 details the FEA results collected in the six test categories set forth above for the VMS and PEEQ %. For example, one preferred prototype, C36, shows a PEEQ expansion and a VMS for stent 1 and a PEEQ and VMS for stent 2. These design results are well within desired criteria to achieve the target of high radial strength. The limiting factor to high radial strength is the limit of the material. Design modifications, such as inner arc radius modification in conjunction with the offset inner arc, impact the PEEQ strain of the stent allowing high radial strength designs within allowable material stresses and strains.

TABLE 3

| | | C30¹ | | C36¹ | | C36² | | C33¹ | | C40¹ | | C41¹ | | C44¹ | |
|---|---|---|---|---|---|---|---|---|---|---|---|---|---|---|---|
| | | VMS | PEEQ | VMS | PEEQ | VMS | PEEQ | VMS | PEEQ | VMS | PEEQ | VMS | PEEQ | VMS | PEEQ |
| Step # | Description | (ksi) | (%) | (ksi) | (%) | (ksi) | (%) | (ksi) | (%) | (ksi) | (%) | (ksi) | (%) | (ksi) | (%) |
| 1 | Crimp | 173 | 27.9 | 176 | 27.6 | 172 | 26.4 | 179 | 28.8 | 180 | 28.9 | 180 | 30.0 | 182 | 29.9 |
| 2 | Recoil | 138 | | 147 | | 145 | | 143 | | 145 | | 151 | | 147 | |
| 3 | Expansion | 350 | 96.3 | 350 | 90.4 | 346 | 87.7 | 350 | 97.2 | 331 | 82.1 | 345 | 87.2 | 344 | 83.2 |
| | Stent 1 | | | | | | | | | | | | | | |
| 4 | Recoil | 183 | | 124 | | 178 | | 181 | | 178 | | 187 | | 180 | |
| 5 | Expansion | 346 | 96.3 | 319 | 90.4 | 334 | 87.7 | 345 | 97.2 | 344 | 86.9 | 340 | 87.2 | 331 | 83.2 |
| | Stent 2 | 350 | (ST1) | 350 | (ST1) | 348 | (ST1) | 350 | (ST1) | 339 | (ST1) | 345 | (ST1) | 338 | (ST1) |
| 6 | Recoil | 181 | 97.8 | 206 | 92.7 | 188 | 88.3 | 196 | 99.0 | 174 | 85.2 | 209 | 87.4 | 180 | 84.7 |
| | | 216 | (ST2) | 236 | (ST2) | 235 | (ST2) | 222 | (ST2) | 209 | (ST2) | 233 | (ST2) | 211 | (ST2) |

As shown in Table 4 and Table 5, the C36 prototype stent of the invention has acceptable FEA data relative to desired PEEQ and VMS.

TABLE 4

| | | | Strut | | | | | | |
|---|---|---|---|---|---|---|---|---|---|
| | | | Width (μm) | | | | | | |
| | | | 2-Link Body | | Metal-to- | | | | |
| | | | Ring Crest, | Rings in | Artery | Labeled | Theoretical | Total | Abluminal |
| | Target | | Bar-Arm, | 18 mm | Ratio | Max Post | Max | Surface | Surface |
| Stent | Thickness | | Link, Non- | Length | Diameter | Dilatation | Expansion | Area | Area |
| Design | (μm) | Description | LinearLink | Design | at 3.0 mm | (mm) | (mm) | (mm2) | (mm2) |
| C36 | 81 | 6 crest | 125 CW, | 13 | 15.3% | 3.75 | 4.3 | 90.6 | 28.41 |
| | | 2 link | 145 BA, | | (+15.0%) | | | (+7.1%) | (+18.6%) |
| | | Distal | 100 LL, | | | | | | |
| | | 6 crest | 80 NLL | | | | | | |
| | | 3 link | | | | | | | |
| | | Proximal | | | | | | | |

TABLE 5

| | FEA @ +/− | FEA @ +/− | |
|---|---|---|---|
| Design | 7 μm Width | 5 μm Width | Radial Strength |
| C30 | Exceed desired limit | Exceed desired limit | ~2200 mmHG |
| C36 | Exceed desired limit | Within desired limit | ~1950 mmHG |
| C46 | Within desired limit | Within desired limit | ~1800 mmHG |

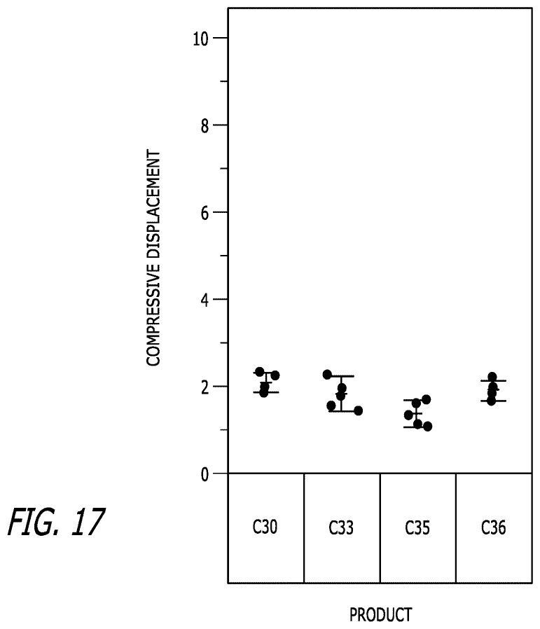

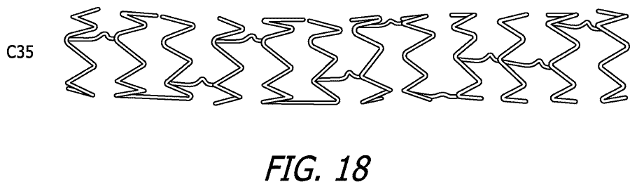

The longitudinal compression of the stents disclosed herein were tested and analyzed as shown in Table 6 and FIGS. 17 and 18. The longitudinal compression testing was conducted in air. The test moves the head of an Instron downward at a speed of 0.2 mm per second onto a stent in a longitudinal configuration and continues to press down on the stent until a force of 0.5N is detected. At this point, the test is stopped, and the compression displacement of the stent is determined. 0.5N is a clinically relevant longitudinal compression force to which a deployed stent may be subjected when the tip of a device is caught while trying to cross a freshly deployed stent.

As shown in Table 6, the longitudinal compression displacement is low for the prototypes of the invention. In FIG. 17, the compressive displacement of the prototype stents is quite low and reflects the higher radial strength incorporated into the stents by moving the inner arc radius to provide an increased non-uniform crest width as described herein. In other words, the offset inner arcs of the prototype stents have a higher radial strength and consequently a higher resistance to longitudinal stent compression. As an example, the C35 prototype stent has an average displacement of 1.4 mm from the longitudinal compression tests. FIG. 18 graphically shows the spacing between rings of the C35 stent design begins to overlap, when the stent is subjected to the longitudinal compression test, and the overlap of the C35 stent is well within acceptable parameters.

TABLE 6

| | | Average | | | | |
|---|---|---|---|---|---|---|
| | | Displacement | | | | |
| Stent Design | Size (mm) | (mm) | Stdev | Min | Max | N |
| C30 13 ring, | 3.0 × 18 | 2.2 | 0.21 | 1.92 | 2.39 | 4 |
| 125CSW, | | | | | | |
| 145BASW | | | | | | |
| C33 12 ring, | 3.0 × 18 | 1.8 | 0.33 | 1.46 | 2.31 | 5 |
| 135CSW, | | | | | | |
| 145BASW | | | | | | |
| C35 12 Ring | 3.0 × 18 | 1.4 | 0.27 | 1.12 | 1.72 | 5 |
| (NLL change) | | | | | | |

In Table 6, CSW stands for crest strut width, BASW stands for bar arm strut width, and NLL stands for non-linear link.

Since many commercial stents have a drug coating (as further discussed herein), the surface area of the stent is a critical feature. The stent of the present invention has a higher overall surface area and higher abluminal surface areas than some commercial stents. As shown in Table 7, the prototype stents of the invention have more favorable metal to artery ratio, total surface area, and allowable surface area than commercial stents. For drug eluting stents (DES), these design features are critical to patient safety and treatment. Higher surface areas means more drugs can be coated on the stent.

TABLE 7

| | | | | Metal-to- | | |
|---|---|---|---|---|---|---|
| | | | | Artery Ratio | Total | Abluminal |
| | | Strut Width (μm): | | at 3.0 mm | Surface | Surface |
| | | 2-Link Body Ring | | Exapnsion | area (mm²) | area (mm²) |
| Design | Description | Crest, Bar-Arm | # Ring | Diameter) | (Δ to A | (Δ to A) |
| C30 | 6 crest 2 link with 6 | 125 Crest width, | 13 | 16.5% | 91.1 | 28.4 |
| | crest 3 link proximal | 145 Bar Arm Width | | | (+7.7%) | (+19.8%) |
| C33 | 6 crest 2 link 6 C 3L | 135 Crest width, | 12 | 11.3% | 84.6 | 26.5 |
| | Prox. | 145 Bar Arm Width | | | (0.0%) | (+11.8%) |
| | C30 + um inner crest | | | | | |
| | conehead -a | | | | | |
| | fine + ring spacing | | | | | |
| C40 | 6 crest 2 link, 6 C 3L | 125 Crest Width, | 13 | 14.3% | 89.4 | 27.8 |
| | Prox. Based on C30 | 145 Bar Arm Width | | | (+5.7%) | (+17.3%) |
| C46 | 6 crest 2 link 6 C 3L | 125 Crest width, | 13 | 15.0% | 90.4 | 28.0 |
| | Prox. | 145 Bar Arm Width | | | (+6.9%) | (+18.1%) |

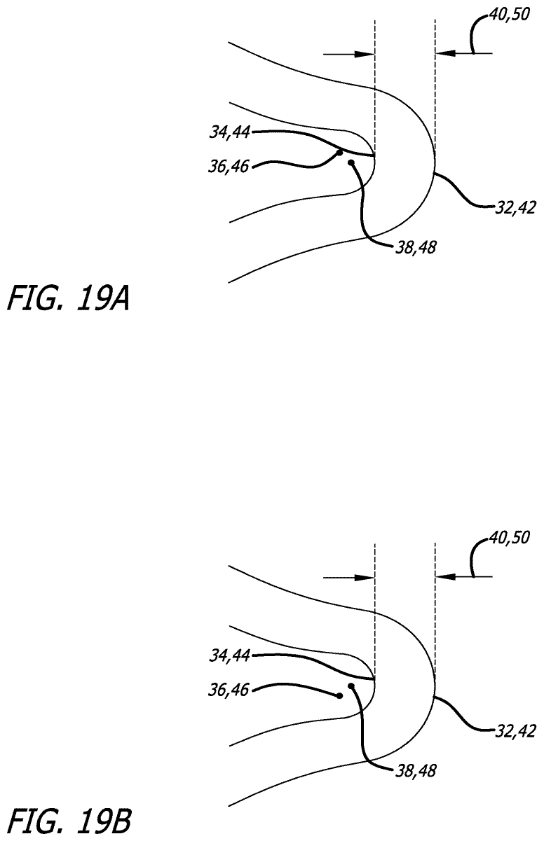

In all of the foregoing embodiments, the center point 36,46 of the inner arc 34,44 is offset from a center point 38,48 of the outer arc 32,42 (respectively) in a direction away from the inner arc 34,44, thereby forming an increased non-uniform crest width 40,50 (respectively). The center point 36,46 of the inner arc 34,44 can be offset in two directions without departing from the invention. As shown in FIG. 19A, the center point 36,46 of the inner arc 34,44 is offset in a direction away from and upward (in FIG. 19A) from the inner arc 34,44. By way of example, the center point 36,46 is moved between 2 μm and 10 μm away from and between 1 μm and 8 μm upwards from the inner arc 34,44 (FIG. 19A). In FIG. 19B, the center point 36,46 is moved between 2 μm and 10 μm away from, and between 1 μm and 8 μm downwards from the inner arc 34,44. The increase in radial strength in these embodiments is comparable to that disclosed in the embodiments described in FIGS. 7-18.

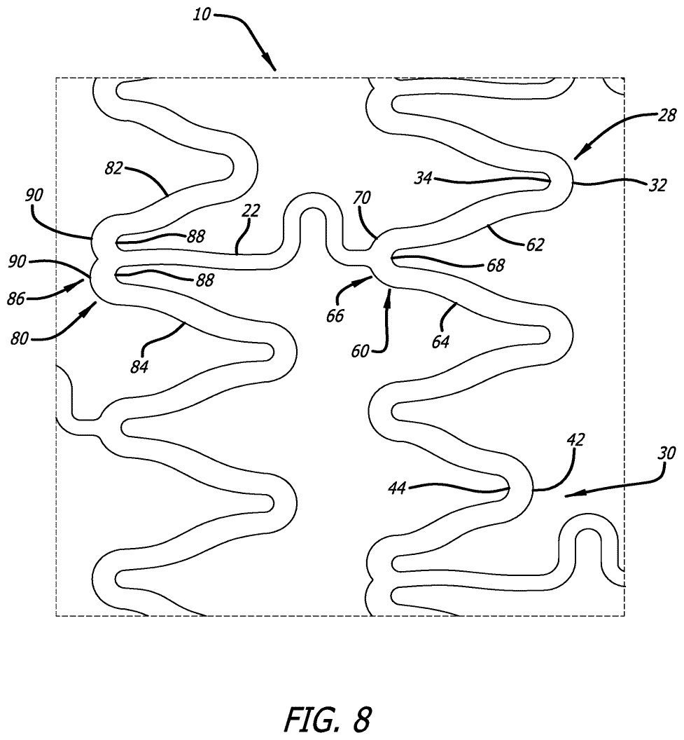

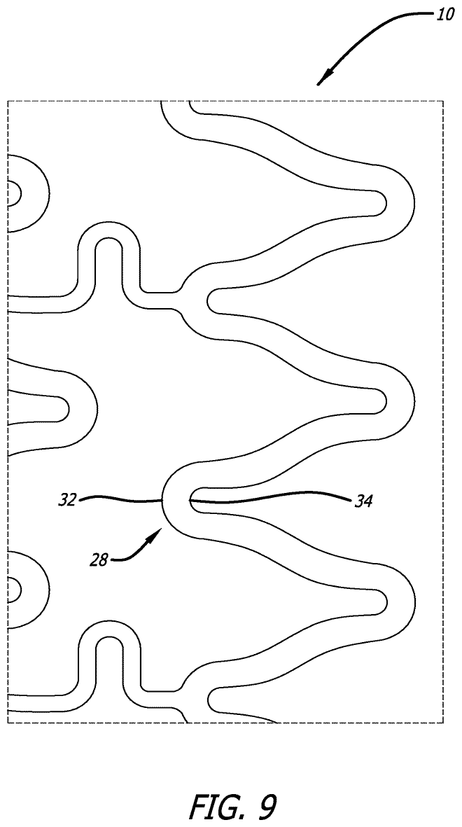

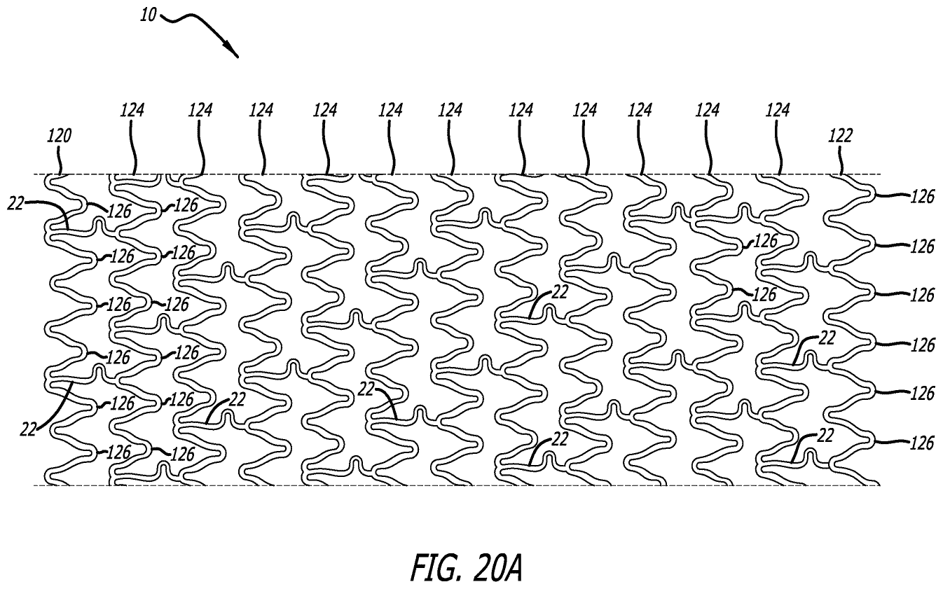

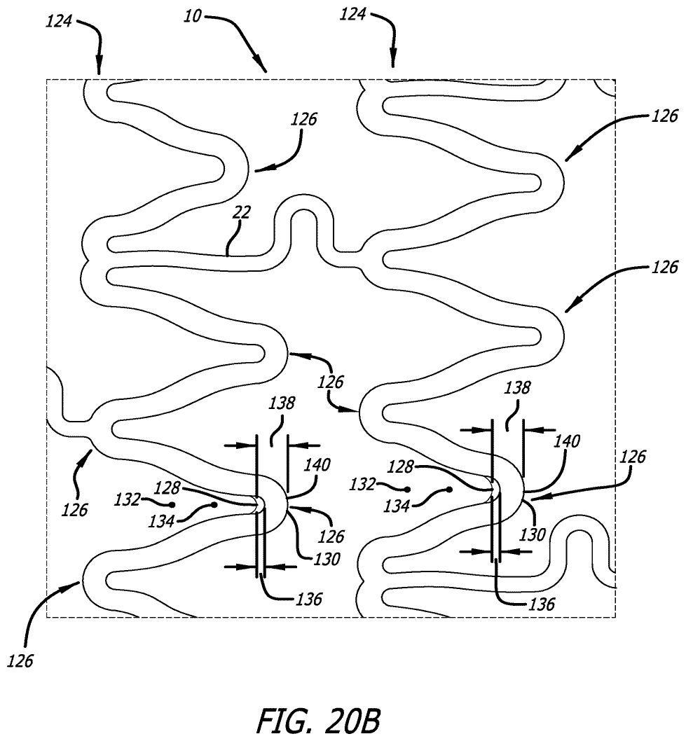

In another embodiment, shown in FIGS. 20A and 20B, a stent 10 has the same structural features of stent 10 shown in FIG. 8. In this embodiment, a distal end ring 120, a proximal end ring 122, and a plurality of body rings 124 therebetween, each have a plurality of crests 126. In order to increase the radial strength, each of the plurality of crests 126 has an inner arc 128 and an outer arc 130, wherein a centerpoint 132 of the inner arc 128 is offset from a center point 134 of the outer arc 130 in a direction away from the inner arc 128, thereby forming an increased non-uniform width 136 on the plurality of crests 126. The increased non-uniform width 136 on the plurality of crests 126 results in an increased strut width 136 at an apex 140 of the plurality of crests 126, and a corresponding increase in radial strength of the distal end ring 120, proximal end ring 122, and the plurality of body rings 124. The variation in offset between the centerpoint 132 of the inner arc 128 and the center point 134 of the outer arc 130 is in a range from 2 μm to 100 μm. The strut width 138 at the apex 140 is in a range from 40 μm to 250 μm. All of the rings of this embodiment are connected by links 22 in a pattern described above, such as 2-2-N-3-3, where the Nth link represents any number of 2-link connections between adjacent rings necessary to form a stent 10 having a length in a range from 18 mm to 58 mm.

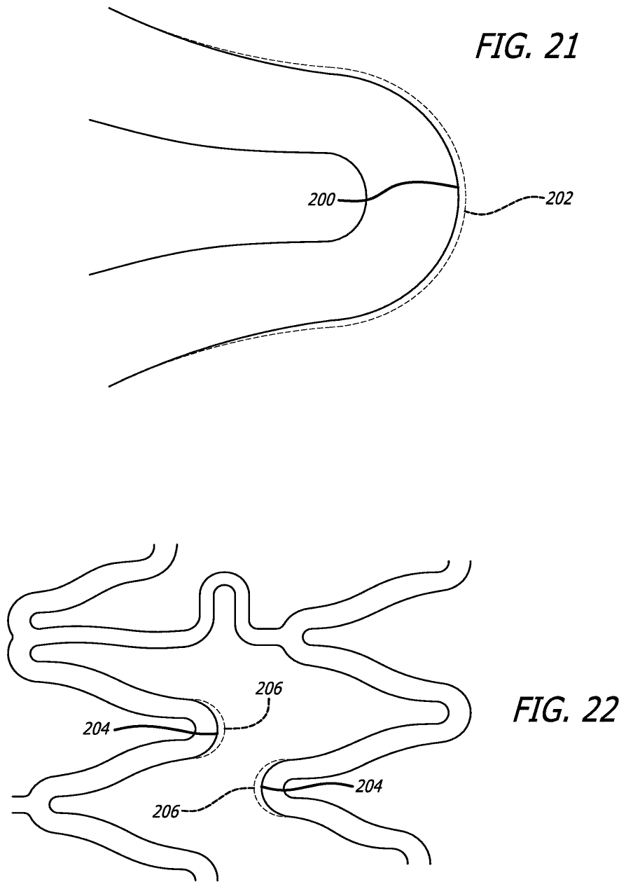

It is well known in the art of stent designing that crest dimensions affect radial strength. For cases where increasing crest thickness to increase radial strength is not desired, crest strut width is often increased to increase radial strength. However, increasing crest strut width while keeping crest inner radius the same always results in an increase to the crest profile as illustrated in FIG. 21, with the solid arc line 200 representing the unmodified crest and the dashed arc line 202 representing the wider crest strut width. In this example, the crest profile increased from 0.36 mm to 0.38 mm. This increase to the crest profile consequently results in an increase to the overall stent profile.

For cases where increasing the overall stent profile is not desired, a conehead can be added to the outer crest (hereafter referred to as outer conehead) to increase the strut width only at the apex of the crest while keeping the crest profile unchanged. FIG. 22 illustrates this concept which can be applied to any crests in a stent design. The solid arc line 204 on the outer crest represents the original crest, and the dashed arc line 206 on the outer crest represents the outer conehead, in this example, adding 0.01 mm to the crest strut width at the apex. However, adding outer coneheads to crests decreases spacing in the longitudinal direction between crests in one ring and crests in adjacent ring (hereafter referred to as ring spacing) as illustrated in FIG. 22 (in this example, ring spacing decreased from 0.25 mm to 0.23 mm). In addition, adding outer coneheads to crests results in the inner arc and outer arc of the crest not having the same center point. Instead, their center points are separated by a distance equal to the increase to the crest strut width at the apex. In this example, the separation of center points is 0.01 mm as illustrated in FIG. 22.

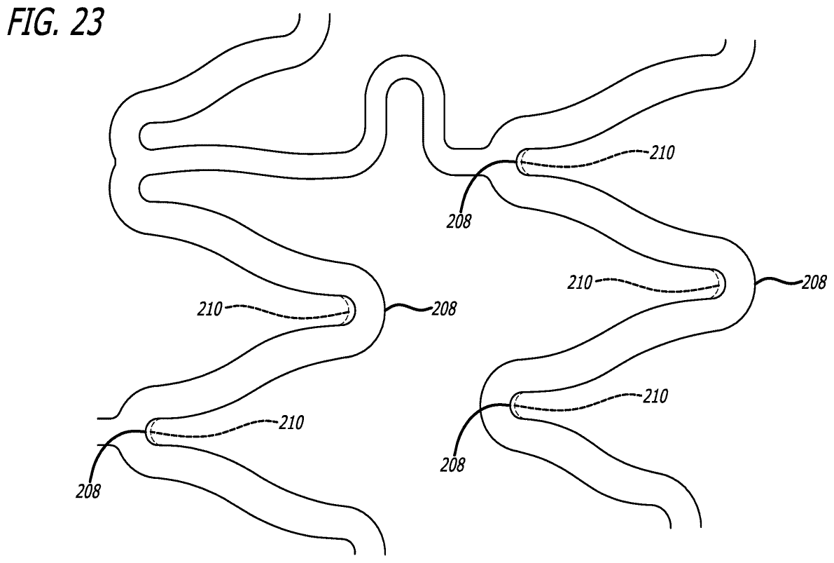

For cases where decreasing ring spacing in a stent design and increasing the overall stent profile are both not desired, a conehead can be added to the inner crest (hereafter referred to as inner conehead) to increase the strut width only at the apex of the crest while keeping the crest profile and ring spacing unchanged. FIG. 23 illustrates this concept which can be applied to any crests in a stent design. The solid arc line 208 on the inner crest represents the original crest, and the dashed arc line 210 on the inner crest represents the inner conehead, in this example, adding 0.01 mm to the crest strut width at the apex. Adding inner coneheads to crests results in the inner arc and outer arc of the crest not having the same center point. Instead, their center points are separated by a distance equal to the increase to the crest strut width at the apex. In this example, the separation of center points is 0.01 mm as illustrated in FIG. 23 below.

In one embodiment of the present invention, inner coneheads were incorporated into the crests of the entire stent to increase its radial strength without increasing its overall profile or decreasing its ring spacing. Table 8 details the strut width dimensions for the crest apex, bar arm, linear link, and non-linear link of C32 (an embodiment without inner coneheads) and C33 (C32 with 0.01 mm inner coneheads) and the overall profiles of both embodiments. As observed in Table 8, the crest apex strut widths of C33 are 0.010 mm larger than those of C32 while all other dimensions including the overall profile remained unchanged. Both embodiments have a strut thickness of 0.081 mm.

TABLE 8
C32 and C33 Strut Width Dimensions

| | | | | | Non- | |
|---|---|---|---|---|---|---|
| | | Crest | Bar Arm | Linear | Linear | |
| | | Strut | Strut | Link Strut | Strut | |
| Stent | | Width | Width | Width | Width | Profile |
| Design | Ring | (mm) | (mm) | (mm) | (mm) | (mm) |
| C32 | 6-crest | 0.125 | 0.145 | 0.100 | 0.090 | 0.795 |
| | 2-link | | | | | |
| | 6-crest | 0.110 | 0.130 | 0.090 | 0.080 | 0.792 |
| | 3-link | | | | | |
| C33 | 6-crest | 0.135 | 0.145 | 0.100 | 0.090 | 0.795 |
| | 2-link | | | | | |
| | 6-crest | 0.120 | 0.130 | 0.090 | 0.080 | 0.792 |
| | 3-link | | | | | |

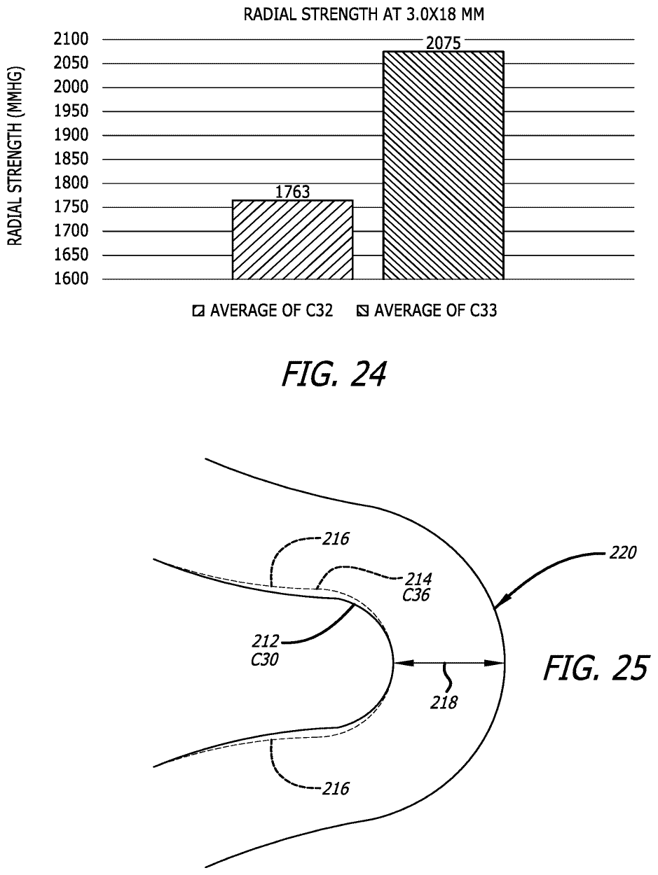

FIG. 24 illustrates the radial strengths of C32 and C33 which have a 3.0 mm diameter and an 18 mm length. An increase in radial strength is observed from C32 to C33 as a result of adding 0.01 mm inner coneheads to all crests of C33.

It is well known in the art of stent designing that crest dimensions affect radial strength, and stent designs with wider strut width and thicker strut thickness at crests would have high radial strength. For cases where increasing strut thickness is not an option, crest strut width would be the only dimension optimized for high radial strength. However, this method often results in high stress and strain at the crests at expansion. It is also well known in the art of stent designing that increasing crest inner radius while leaving the outer arc of the crest unchanged would reduce stress and strain at expansion and also reduce radial strength. To reduce stress and strain at the crests without significant impact on radial strength, one embodiment of the present invention reduces the inner crest radii to reduce stress and strain at expansion and then adds inner coneheads of amount equal to the crest inner radius increase to the crests to maintain the strut width at the crest apex to reduce the impact of this modification on radial strength. This embodiment having is illustrated in FIG. 25 and compares the design of the embodiment C30 crest solid inner arc line 212 to the design of the embodiment C36 crest embodiment dashed inner arc line 214. Compared to the C30 crest, the C36 crest carves out the inner arc line 214 on the strut sides 216 while maintaining the strut width 218 at the crest apex 220. The effectiveness of this crest modification on stress and strain and radial strength can be observed in Table 9.

TABLE 9
C30 and C36 Comparison

| | Stress (Ksi) at 3.75 mm | Strain (%) at 3.75 mm | Radial |
|---|---|---|---|
| Stent | Inner Diameter | Inner Diameter | Strength |
| Design | Expansion | Expansion | (mmHg) |
| C30 | >350 | 96.3 | 2222 |
| C36 | 346 | 87.7 | 1994 |

One important feature of all of the embodiments of the present invention is the capability of the stents to expand from a low-profile diameter to a diameter much greater than heretofore was available, while still maintaining structural integrity in the expanded state and remaining highly flexible. Due to the novel structures, the stents of the present invention can have an overall expansion ratio of about 1.0 up to about 5.0 times the original diameter, or more, using certain compositions of stainless steel or cobalt chrome. For example, a 316L stainless steel stent or L605 cobalt chrome stent of the invention can be radially expanded from a diameter of 1.2 mm up to a diameter of about 5.75 mm, which deforms the structural members beyond the elastic limit. The stents still retain structural integrity in the expanded state and will serve to hold open the vessel in which they are implanted. Materials other than stainless steel (316L) may afford higher or lower expansion ratios without sacrificing structural integrity.

The stents of the present invention can be made in many ways. The preferred method of making the stent is to cut a thin-walled tubular member, such as a stainless steel or cobalt chrome tubing, to remove portions of the tubing in the desired pattern for the stent, leaving relatively untouched the portions of the metallic tubing which are to form the stent. It is preferred to cut the tubing in the desired pattern by means of a machine-controlled laser which is well known in the art. Electropolishing the stent is also well known in the art.

The stent tubing may be made of a suitable biocompatible material such as stainless steel, titanium, cobalt-chromium, tantalum, super-elastic (nickel-titanium) NiTi alloys and even high strength thermoplastic polymers. When stainless steel is utilized, the stainless steel can be one-eighth hardened due to a straightening process and then annealed to make the stent plastically deformable to thus remove intrinsic recoil post deployment. The stent diameters are very small, so the tubing from which it is made must necessarily also have a small diameter. For stents implanted in other body lumens, such as PTA applications in larger vessels like the renal artery, the dimensions of the tubing are correspondingly larger. The diameters and tubing wall thickness of the stents can vary according to a particular application and are known in the art. While it is preferred that the stents be made from laser cut tubing, those skilled in the art will realize that the stent can be laser cut from a flat sheet and then rolled up in a cylindrical configuration with the longitudinal edges welded or similarly joined to form a cylindrical shape.

The stents may also be made of materials such as superelastic (sometimes called pseudo-clastic) nickel-titanium (NiTi) alloys. In this case, the stent would be formed full size but deformed diametrically (e.g. compressed) to a smaller diameter onto the delivery catheter to facilitate intraluminal delivery to a desired intraluminal site. The stress induced by the deformation transforms the stent from an austenite phase to a martensite phase to enable the compression into a capture sheath of the delivery catheter, and upon release of the compressive pressure when the stent reaches the desired intraluminal location, allows the stent to fully expand into the vessel due to the transformation of the nitinol back to the more stable austenite phase.

The present invention stent is ideally suited for drug delivery (i.e., delivery of a therapeutic agent) since it has a relatively uniform ratio of stent versus open surface area which ensures uniform distribution of drugs delivered within the vessel. Typically, a polymer is coated onto the stent of the type disclosed in U.S. Pat. Nos. 6,824,559 and 6,783,793 which are incorporated herein by reference.

These bioactive agents can be any agent, which is a therapeutic, prophylactic, or diagnostic. These agents can have anti-proliferative or anti-inflammatory properties or can have other properties such as antineoplastic, antiplatelet, anti-coagulant, anti-fibrin, antithrombonic, antimitotic, antibiotic, antiallergic, antioxidant as well as cystostatic agents. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Nucleic acid sequences include genes, antisense molecules which bind to complementary DNA to inhibit transcription, and ribozymes. Some other examples of other bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of anti-proliferative agents include rapamycin and its functional or structural derivatives, 40-O-(2-hydroxy) ethyl-rapamycin (everolimus), and its functional or structural derivatives, paclitaxel and its functional and structural derivatives. Examples of rapamycin derivatives include methyl rapamycin, ABT-578 (Zotarolimus), 40-O-(3-hydroxy) propyl-rapamycin, 40-O-[2-(2-hydroxy)ethoxy]ethyl-rapamycin, and 40-O-tetrazole-rapamycin. Examples of paclitaxel derivatives include docetaxel. Examples of antincoplastics and/or antimitotics include methotrexate, azathioprine, vincristine, vinblastine, fluorouracil, doxorubicin hydrochloride (e.g. ADRIAMYCIN® from Pharmacia & Upjohn, Peapack N.J.), and mitomycin (e.g. MUTAMYCIN® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax a (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-COA reductase, a cholesterol lowering drug, brand name MEVACOR® from Merck & Co., Inc., Whitehouse Station, N.J.), monoclonal antibodies (such as those specific for Platelet-Derived Growth Factor (PDGF) receptors), nitroprus side, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of anti-inflammatory agents including steroidal and non-steroidal anti-inflammatory agents include tacrolimus, dexamethasone, clobetasol, combinations thereof. Examples of such cytostatic substance include angiopeptin, angiotensin converting enzyme inhibitors such as captopril (e.g. CAPOTEN® and CAPOZIDE® from Bristol-Myers Squibb Co., Stamford, Conn.), cilazapril or lisinopril (e.g. PRINIVIL® and PRINZIDE® from Merck & Co., Inc., Whitehouse Station, N.J.). An example of an antiallergic agent is permirolast potassium. Other therapeutic substances or agents which may be appropriate include alpha-interferon, bioactive RGD, and genetically engineered epithelial cells. The foregoing substances can also be used in the form of prodrugs or co-drugs thereof. The bioactive agents also include metabolites of the foregoing substances and prodrugs of these metabolites. The foregoing substances are listed by way of example and are not meant to be limiting. Other active agents which are currently available or that may be developed in the future are equally applicable.

While the invention has been illustrated and described herein in terms of its use as intravascular stents, it will be apparent to those skilled in the art that the stents can be used in other instances in all vessels in the body. Since the stents of the present invention have the novel feature of expanding to very large diameters while retaining their structural integrity, they are particularly well suited for implantation in almost any vessel where such devices are or may be used. This feature, coupled with limited longitudinal contraction (i.e., stent length change or foreshortening) of the stent when it is radially expanded, provides a highly desirable support member for all vessels in the body. Other modifications and improvements may be made without departing from the scope of the invention.

I claim:

1. A stent, comprising:
a tubular body having a distal end ring, a proximal end ring, and a plurality of body rings therebetween;
the distal end ring, the proximal end ring, and the plurality of body rings being aligned in an in-phase configuration;
the proximal end ring being connected to a first proximal body ring by three links, and the first proximal body ring being connected to a second proximal body ring by three links;
the distal end ring and the plurality of body rings up to the second proximal body ring being connected by two links;
each of the distal end ring, the proximal end ring, and the plurality of body rings has six crests;
each of the distal end ring and the plurality of body rings up to the second proximal body ring has four long crests and two short crests; and
the four long crests having an outer arc and an inner arc, a center point of the inner arc being offset from a center point of the outer arc in a direction away from the inner arc, thereby forming a non-uniform width on the four long crests.

2. The stent of claim 1, wherein the two short crests having an outer arc and an inner arc, a center point of the inner arc being offset from a center point of the outer arc in a direction away from the inner arc, thereby forming a non-uniform width on the short crests.

3. The stent of claim 1, wherein the proximal end ring has six equal length crests.

4. The stent of claim 3, wherein the six equal length crests on the proximal end ring have an outer arc and an inner arc, a center point of the inner arc being offset from a center point of the outer arc in a direction away from the inner arc, thereby forming a non-uniform width on the six equal length crests.

5. The stent of claim 1, wherein the number of links connecting the rings has a pattern of 2-2-2-2-2-2-2-2-2-2-3-3 moving from the distal end ring toward the proximal end ring.

6. The stent of claim 1, wherein the number of links connecting the rings has a pattern of 2-2-2-2-2-2-2-2-2-3-3 moving from the distal end ring toward the proximal end ring.

7. The stent of claim 1, wherein the number of links connecting the rings has a pattern of 2-2-2-2-2-2-2-2-3-3 moving from the distal end ring toward the proximal end ring.

8. The stent of claim 1, wherein the number of links connecting the rings has a pattern of 2-2-2-2-2-2-2-3-3 moving from the distal end ring toward the proximal end ring.

9. The stent of claim 1, wherein the number of links connecting the rings has a pattern of 2-2-2-2-2-2-3-3 moving from the distal end ring toward the proximal end ring.

10. The stent of claim 1, wherein the number of links connecting the rings has a pattern of 2-2-2-2-2-3-3 moving from the distal end ring toward the proximal end ring.

11. The stent of claim 1, wherein the variation in offset between the center point of the inner arc and the center point of the outer arc is in a range from 2 μm to 100 μm.

12. The stent of claim 11, wherein the inner arc and the outer arc have an arc configuration of a semi-circle arc, or a polyline arc, or a 3-point arc.

13. The stent of claim 12, wherein a crest width of the four long crests and the two short crests is in a range from 40 μm to 250 μm.

14. The stent of claim 1, wherein each of the distal end ring and plurality of body rings has at least two W-shaped crests.

15. The stent of claim 14, wherein the at least two W-shaped crests have an outer arc and an inner arc, a center point of the inner arc being offset from a center point of the outer arc in a direction away from the inner arc, thereby forming a non-uniform width on the at least two W-shaped crests.

16. The stent of claim 15, wherein the variation in offset between the center point of the inner arc and the center point of the outer arc is in a range from 2 μm to 100 μm.

17. The stent of claim 16, wherein the inner arc and the outer arc have an arc configuration of a semi-circle arc, or a polyline arc, or a 3-point arc.

18. The stent of claim 17, wherein a crest width of the W-shaped crests is in a range from 40 μm to 250 μm.

19. The stent of claim 1, wherein each of the proximal end ring and the plurality of body rings have at least two Y-shaped crests.

20. The stent of claim 19, wherein the at least two Y-shaped crests have an outer arc and an inner arc, a center point of the inner arc being offset from a center point of the outer arc in a direction away from the inner arc, thereby forming a non-uniform width on the at least two Y-shaped crests.

21. The stent of claim 20, wherein the variation in offset between the center point of the inner arc and the center point of the outer arc is in a range from 2 μm to 100 μm.

22. The stent of claim 21, wherein the inner arc and the outer arc have an arc configuration of a semi-circle arc, or a polyline arc, or a 3-point arc.

23. The stent of claim 22, wherein a crest width of the Y-shaped crests is in a range from 40 μm to 250 μm.

24. A stent, comprising:
a tubular body having a distal end ring, a proximal end ring, and a plurality of body rings therebetween;
the distal end ring, the proximal end ring, and the plurality of body rings being aligned in an in-phase configuration;
the proximal end ring being connected to a first proximal body ring by three links, and the first proximal body ring being connected to a second proximal body ring by three links;
the distal end ring and the plurality of body rings up to the second proximal body ring being connected by two links;
each of the distal end ring, the proximal end ring, and the plurality of body rings has six crests;
each of the distal end ring and the plurality of body rings up to the second proximal body ring has four long crests and two short crests;
each of the proximal end ring and the plurality of body rings has a Y-shaped member;
the four long crests having an outer arc and an inner arc, a center point of the inner arc being offset from a center point of the outer arc in a direction away from the inner arc, thereby forming a non-uniform width on the four long crests; and
each Y-shaped member has an outer arc and an inner arc, a center point of the inner arc being offset from a center point of the outer arc in a direction away from the inner arc, thereby forming a non-uniform crest width on the Y-shaped members.

25. The stent of claim 24, wherein the two short crests having an outer arc and an inner arc, a center point of the inner arc being offset from a center point of the outer arc in a direction away from the inner arc, thereby forming a non-uniform width on the short crests.

26. The stent of claim 24, wherein the proximal end ring has six equal length crests.

27. The stent of claim 26, wherein the six equal length crests on the proximal end ring have an outer arc and an inner arc, a center point of the inner arc being offset from a center point of the outer arc in a direction away from the inner arc, thereby forming a non-uniform width on the six equal length crests.

28. The stent of claim 24, wherein the number of links connecting the rings has a pattern of 2-2-2-2-2-2-2-2-2-2-3-3 moving from the distal end ring toward the proximal end ring.

29. The stent of claim 24, wherein the number of links connecting the rings has a pattern of 2-2-2-2-2-2-2-2-2-3-3 moving from the distal end ring toward the proximal end ring.

30. The stent of claim 24, wherein the number of links connecting the rings has a pattern of 2-2-2-2-2-2-2-2-3-3 moving from the distal end ring toward the proximal end ring.

31. The stent of claim 24, wherein the number of links connecting the rings has a pattern of 2-2-2-2-2-2-2-3-3 moving from the distal end ring toward the proximal end ring.

32. The stent of claim 24, wherein the number of links connecting the rings has a pattern of 2-2-2-2-2-2-3-3 moving from the distal end ring toward the proximal end ring.

33. The stent of claim 24, wherein the number of links connecting the rings has a pattern of 2-2-2-2-2-3-3 moving from the distal end ring toward the proximal end ring.

34. The stent of claim 24, wherein the variation in offset between the center point of the inner arc and the center point of the outer arc is in a range from 2 μm to 100 μm.

35. The stent of claim 34, wherein the inner arc and the outer arc have an arc configuration of a semi-circle arc, or a polyline arc, or a 3-point arc.

36. The stent of claim 35, wherein a crest width of the four long crests and the two short crests is in a range from 40 μm to 250 μm.

37. The stent of claim 24, wherein each of the distal end ring and the plurality of body rings has at least two W-shaped crests.

38. The stent of claim 37, wherein the at least two W-shaped crests have an outer arc and an inner arc, a center point of the inner arc being offset from a center point of the outer arc in a direction away from the inner arc, thereby forming a non-uniform width on the at least two W-shaped crests.

39. The stent of claim 38, wherein the variation in offset between the center point of the inner arc and the center point of the outer arc is in a range from 2 μm to 100 μm.

40. The stent of claim 39, wherein the inner arc and the outer arc have an arc configuration of a semi-circle arc, or a polyline arc, or a 3-point arc.

41. The stent of claim 40, wherein a crest width of the W-shaped crests is in a range from 40 μm to 250 μm.

42. The stent of claim 24, wherein the variation in offset between the center point of the inner arc and the center point of the outer arc is in a range from 2 μm to 100 μm.

43. The stent of claim 42, wherein the inner arc and the outer arc have an arc configuration of a semi-circle arc, or a polyline arc, or a 3-point arc.

44. The stent of claim 43, wherein a crest width of the Y-shaped members is in a range from 40 μm to 250 μm.

45. A stent, comprising:
a tubular body having a distal end ring, a proximal end ring, and a plurality of body rings therebetween;
the distal end ring, the proximal end ring, and the plurality of body rings being aligned in an in-phase configuration;
the proximal end ring being connected to a first proximal body ring by three links, and the first proximal body ring being connected to a second proximal body ring by three links;
the distal end ring and the plurality of body rings up to the second proximal body ring being connected by two links;
each of the distal end ring, the proximal end ring, and the plurality of body rings has six crests;
each of the distal end ring and the plurality of body rings has four long crests and two short crests; and
each of the distal end ring and the plurality of body rings have a W-shaped member; and
each W-shaped member has an outer arc and an inner arc, a center point of the inner arc being offset from a center point of the outer arc in a direction away from the inner arc, thereby forming a non-uniform crest width on the W-shaped members.

46. The stent of claim 45, wherein:
the four long crests having an outer arc and an inner arc, a center point of the inner arc being offset from a center point of the outer arc in a direction away from the inner arc, thereby forming a non-uniform width on the four long crests.

47. The stent of claim 45, wherein the two short crests having an outer arc and an inner arc, a center point of the inner arc being offset from a center point of the outer arc in a direction away from the inner arc, thereby forming a non-uniform width on the two short crests.

48. The stent of claim 45, wherein the proximal end ring has six equal length crests.

49. The stent of claim 48, wherein the six equal length crests on the proximal end ring have an outer arc and an inner arc, a center point of the inner arc being offset from a center point of the outer arc in a direction away from the inner arc, thereby forming a non-uniform width on the six equal length crests.

50. The stent of claim 45, wherein the number of links connecting the rings has a pattern of 2-2-2-2-2-2-2-2-2-2-3-3 moving from the distal end ring toward the proximal end ring.

51. The stent of claim 45, wherein the number of links connecting the rings has a pattern of 2-2-2-2-2-2-2-2-2-3-3 moving from the distal end ring toward the proximal end ring.

52. The stent of claim 45, wherein the number of links connecting the rings has a pattern of 2-2-2-2-2-2-2-2-3-3 moving from the distal end ring toward the proximal end ring.

53. The stent of claim 45, wherein the number of links connecting the rings has a pattern of 2-2-2-2-2-2-2-3-3 moving from the distal end ring toward the proximal end ring.

54. The stent of claim 45, wherein the number of links connecting the rings has a pattern of 2-2-2-2-2-2-3-3 moving from the distal end ring toward the proximal end ring.

55. The stent of claim 45, wherein the number of links connecting the rings has a pattern of 2-2-2-2-2-3-3 moving from the distal end ring toward the proximal end ring.

56. The stent of claim 45, wherein the variation in offset between the center point of the inner arc and the center point of the outer arc is in a range from 2 μm to 100 μm.

57. The stent of claim 56, wherein the inner arc and the outer arc have an arc configuration of a semi-circle arc, or a polyline arc, or a 3-point arc.

58. The stent of claim 57, wherein a crest width on the W-shaped crests is in a range from 40 μm to 250 μm.

59. The stent of claim 45, wherein each of the distal end ring and the plurality of body rings has at least two W-shaped crests.

60. The stent of claim 46, wherein the variation in offset between the center point of the inner arc and the center point of the outer arc is in a range from 2 μm to 100 μm.

61. The stent of claim 60, wherein the inner arc and the outer arc have an arc configuration of a semi-circle arc, or a polyline arc, or a 3-point arc.

62. The stent of claim 61, wherein a crest width of the four long crests is in a range from 40 μm to 250 μm.

63. The stent of claim 47, wherein the variation in offset between the center point of the inner arc and the center point of the outer arc is in a range from 2 μm to 100 μm.

64. The stent of claim 63, wherein the inner arc and the outer arc have an arc configuration of a semi-circle arc, or a polyline arc, or a 3-point arc.

65. The stent of claim 64, wherein a crest width of the three short crests is in a range from 40 μm to 250 μm.

66. A stent, comprising:
a tubular body having a distal end ring, a proximal end ring, and a plurality of body rings therebetween;
the distal end ring, the proximal end ring, and the plurality of body rings being aligned in an in-phase configuration;
the proximal end ring being connected to a first proximal body ring by three links, and the first proximal body ring being connected to a second proximal body ring by three links;
the distal end ring and the plurality of body rings up to the second proximal body ring being connected by two links;
each of the distal end ring, the proximal end ring, and the plurality of body rings has six U-shaped members;
each of the distal end ring and the plurality of body rings up to the second proximal body ring has four long U-shaped members and two short U-shaped members; and
the four long U-shaped members having an outer arc and an inner arc, a center point of the inner arc being offset from a center point of the outer arc in a direction away from the inner arc, thereby forming a non-uniform width on the four long U-shaped members.

67. The stent of claim 66, wherein the two short U-shaped members having an outer arc and an inner arc, a center point of the inner arc being offset from a center point of the outer arc in a direction away from the inner arc, thereby forming a non-uniform width on the two short U-shaped members.

68. The stent of claim 65, wherein the proximal end ring has six equal length U-shaped members.

69. The stent of claim 68, wherein the six equal length U-shaped members on the proximal end ring have an outer arc and an inner arc, a center point of the inner arc being offset from a center point of the outer arc in a direction away from the inner arc, thereby forming a non-uniform width on the six equal length U-shaped members.

70. The stent of claim 65, wherein the number of links connecting the rings has a pattern of 2-2-2-2-2-2-2-2-2-2-3-3 moving from the distal end ring toward the proximal end ring.

71. The stent of claim 65, wherein the number of links connecting the rings has a pattern of 2-2-2-2-2-2-2-2-2-3-3 moving from the distal end ring toward the proximal end ring.

72. The stent of claim 65, wherein the number of links connecting the rings has a pattern of 2-2-2-2-2-2-2-2-3-3 moving from the distal end ring toward the proximal end ring.

73. The stent of claim 65, wherein the number of links connecting the rings has a pattern of 2-2-2-2-2-2-2-3-3 moving from the distal end ring toward the proximal end ring.

74. The stent of claim 65, wherein the number of links connecting the rings has a pattern of 2-2-2-2-2-2-3-3 moving from the distal end ring toward the proximal end ring.

75. The stent of claim 65, wherein the number of links connecting the rings has a pattern of 2-2-2-2-2-3-3 moving from the distal end ring toward the proximal end ring.

76. The stent of claim 65, wherein the variation in offset between the center point of the inner arc and the center point of the outer arc is in a range from 2 μm to 100 μm.

77. The stent of claim 76, wherein the inner arc and the outer arc have an arc configuration of a semi-circle arc, or a polyline arc, or a 3-point arc.

78. The stent of claim 77, wherein a crest width of the four long U-shaped members and the two short U-shaped members is in a range from 40 μm to 250 μm.

79. A stent, comprising:
a tubular body having a distal end ring, a proximal end ring, and a plurality of body rings therebetween;
the distal end ring, the proximal end ring, and the plurality of body rings being aligned in an in-phase configuration;
multiple connecting links connect the distal end ring, the plurality of body rings, and the proximal end ring;
each of the distal end ring, the proximal end ring, and the plurality of body rings has a plurality of crests; and
the plurality of crests having an outer arc and an inner arc, a center point of the inner arc being offset from a center point of the outer arc in a direction away from the inner arc, thereby forming an increased non-uniform width on the plurality of crests.

80. The stent of claim 79, wherein the increased non-uniform width on the plurality of crests comprises an increased strut width at an apex of the plurality of crests.

81. The stent of claim 80, wherein at least some of the multiple connecting links have a non-linear configuration.

82. The stent of claim 81, wherein the inner arc and the outer arc have an arc configuration of a semi-circle arc, or a polyene arc, or a 3-point arc.

83. The stent of claim 82, wherein the variation in offset between the center point of the inner arc and the center point of the outer arc is in a range from 2 μm to 100 μm.

84. The stent of claim 82, wherein a strut width of the plurality crests is in a range from 40 μm to 250 μm.

85. The stent of claim 79, wherein the number of links connecting the rings has a pattern of 2-2-2-2-2-2-2-2-2-2-3-3 moving from the distal end ring toward the proximal end ring.

86. The stent of claim 79, wherein the number of links connecting the rings has a pattern of 2-2-2-2-2-2-2-2-2-3-3 moving from the distal end ring toward the proximal end ring.

87. The stent of claim 79, wherein the number of links connecting the rings has a pattern of 2-2-2-2-2-2-2-2-3-3 moving from the distal end ring toward the proximal end ring.

88. The stent of claim 79, wherein the number of links connecting the rings has a pattern of 2-2-2-2-2-2-2-3-3 moving from the distal end ring toward the proximal end ring.

89. The stent of claim 79, wherein the number of links connecting the rings has a pattern of 2-2-2-2-2-2-3-3 moving from the distal end ring toward the proximal end ring.

90. The stent of claim 79, wherein the number of links connecting the rings has a pattern of 2-2-2-2-2-3-3 moving from the distal end ring toward the proximal end ring.

91. The stent of claim 79, wherein the number of links connecting the rings has a pattern of 2-2-N-3-3 moving from the distal end ring toward the proximal end ring.
